(19) United States
(12) Patent Application Publication
(10) Pub. No.: US 2025/0161262 A1
(43) Pub. Date: May 22, 2025

(54) Pharmaceutical Compositions and Methods for Treating Covid

(71) Applicant: Steven BARANOWITZ, Wyncote, PA (US)

(72) Inventor: Steven BARANOWITZ, Wyncote, PA (US)

(21) Appl. No.: 18/729,073

(22) PCT Filed: Jan. 30, 2023
(86) PCT No.: PCT/US2023/011813

§ 371 Date: Jul. 15, 2024

Related U.S. Application Data
(60) Provisional application No. 63/306,148, filed on Feb. 3, 2022.

(51) Int. Cl. A61K 31/365; A61K 31/7088; A61P 31/14

(52) U.S. Cl. CPC A61K 31/365; A61K 31/7088; A61P 31/14

(57) ABSTRACT

Pharmaceutical compositions and methods for preventing, treating, relieving, or ameliorating symptoms of coronavirus infection, including COVID-19 and variants thereof, including treating or preventing severe illness from corona vims infection, comprising the administration of IMPDH inhibitors and/or restricted diets of guanosine-containing nucleosides or nucleotides.

This PCT application claims the benefit under 35 U.S.C. § 119(e) of Application Ser. No. 63/306,148 filed on Feb. 3, 2022, entitled PHARMACEUTICAL COMPOSITIONS AND METHODS FOR TREATING COVID and whose entire disclosure is incorporated by reference herein.

### SPECIFICATION

### Background

Coronaviruses (CoVs) are enveloped viruses with a positive sense single-stranded RNA genome. Four coronavirus genera (alpha, beta, gamma, and delta) have been identified. Examples of beta-coronaviruses include MERS-CoV, SARS-CoV, HCov-OC42, and HCoV-HKUl, and now SARS-CoV2. It is hypothesized that SARS-CoV2 may escape the innate and adaptive immune systems in a manner similar to that of SARS-CoV and MERS-CoV.

SARS-Cov2 infection may lead to COVID-19. As many as 75% of positive cases can be asymptomatic, while the asymptomatic carriers continue spreading the virus. In a majority of cases, signs and symptoms of COVID-19 disease are mild to moderate and resolve. However, in vulnerable populations the disease manifests as an overwhelming and potentially fatal condition. Besides advanced age, predisposing conditions such as hypertension, diabetes, congestive heart failure, obesity, cancers, and immunosuppression, for various reasons, lead to increased intensity of the disease and a poor prognosis. The COVID-19 disease appears to produce runaway inflammation, direct destruction of lung cells involved in gas exchange and their impaired regeneration, and accelerated lung scarring in those seriously afflicted. COVID-19, for example, may initially present with mild, moderate, or severe illness. Patients may initially exhibit a mild disease, presenting with symptoms of an upper respiratory tract viral infection, including mild fever, cough, shortness of breath, sore throat, nasal congestion, malaise, headache, and/or muscle pain. Some patients will progress to moderate or severe pneumonia, and a fraction of patients will progress to Acute Respiratory Distress Syndrome (ARDS) or sepsis or septic shock, which can be life threatening. Some infected individuals lose the ability to smell and/or taste. Other symptoms may include body aches, chills, fatigue, nausea, and diarrhea. COVID-19 symptoms may lead to death, in part, due to complications such as pneumonia and/or organ failure. On the other hand, some people infected with SARS-CoV2 may be asymptomatic. The incubation period for SARS-CoV2 ranges from one to fourteen days, with a median period from five to six days.

Compositions and methods are needed for preventing, treating, relieving, or ameliorating symptoms of coronavirus infection, including SARS-CoV2 and variants thereof, including treating or preventing severe illness from coronavirus infection, such as COVID-19. The present disclosure provides compositions and methods for preventing, treating, relieving, or ameliorating symptoms of coronavirus infection, including COVID-19 and variants thereof, including treating or preventing severe illness from coronavirus infection.

All references cited herein are incorporated herein by reference in their entireties.

### BRIEF SUMMARY

The disclosure provides a treatment method for improving, compared to a baseline measurement, the blood oxygenation level of a subject diagnosed with a viral infection, the treatment method comprising: selecting a subject diagnosed with a viral infection and in need of improving, compared to a baseline measurement, blood oxygenation level; administering to the patient at least one agent which depletes guanosine-containing nucleosides and nucleotides, thereby improving the subject's blood oxygenation level. The disclosure provides a treatment method wherein the at least one agent which depletes guanosine-containing nucleosides and nucleotides is selected from the group consisting of mycophenolate, IMPDH enzyme inhibitors, agents which bind to guanine, or guanosine-containing nucleosides and nucleotides, and combinations thereof. The disclosure provides a treatment method wherein the baseline measurement is determined prior to infection, early in the subject's infection, from a standard, compared to a control group, or an historical control value. The disclosure provides a treatment method wherein the subject is further diagnosed with acute respiratory distress syndrome (ARDS). The disclosure provides a treatment method wherein the blood oxygenation level is determined using the oxygenation index, the oxygenation saturation index, pulse oximetry, and combinations thereof. The disclosure provides a treatment method wherein the blood oxygenation level is determined at a time point selected from the group consisting of one day, two days, three days, four days, five days, six days, seven days, eight days, nine days, 10 days, 11 days, 12 days, 13 days, two weeks, three weeks, one month, two months, and three months. The disclosure provides a treatment method wherein said treatment reduces ventilator use, reduces ECMO use, reduces time in the ICU or CCU, reduces hospitalization time, reduces all-cause mortality, or reduces supplemental oxygen consumption compared to a control group or historical controls. The disclosure provides a treatment method wherein said treatment reduces pulmonary exudate levels or reduces supplemental oxygen consumption compared to a baseline measurement. The disclosure provides a treatment method wherein said treatment increases subject's survival compared to a control group or historical controls. The disclosure provides a treatment method wherein the viral infection is a coronavirus selected from the group consisting of SARS-CoV, HCoV NL63, HKUl, MERS-CoV, and SARS-CoV-2. The disclosure provides a treatment method wherein the coronavirus is SARS-CoV-2.

The disclosure provides a treatment method for improving, compared to a baseline measurement, the blood oxygenation level of a subject diagnosed with a viral infection, the treatment method comprising: selecting a subject diagnosed with a viral infection and in need of improving, compared to a baseline measurement, blood oxygenation level; administering to the patient a diet which depletes guanosine-containing nucleosides and nucleotides; wherein the subject's blood oxygenation level is improved compared to a baseline measurement. The disclosure provide a treatment method wherein the diet which depletes guanosine-containing nucleosides and nucleotides contains a nucleotide content which is selected from the group consisting of about 1000 mg/day, of about 750 mg/day, of about 500 mg/day, of about 250 mg/day, of about 100 mg/day, of about 75 mg/day, of about 50 mg/day, and of about 25 mg/day of nucleotide. The disclosure provides a treatment method wherein the baseline measurement is determined prior to infection, early in the subject's infection, from a standard, compared to a control group, or an historical control value. The disclosure provides a treatment method wherein the subject is further diagnosed with acute respiratory distress syndrome (ARDS). The disclosure provides a treatment method wherein the blood oxygenation level is determined using the oxygenation index, the oxygenation saturation index, pulse oximetry, and combinations thereof.

The disclosure provides a treatment method wherein the blood oxygenation level is determined at a time point selected from the group consisting of one day, two days, three days, four days, five days, six days, seven days, eight days, nine days, 10 days, 11 days, 12 days, 13 days, two weeks, three weeks, one month, two months, and three months. The disclosure provides a treatment method wherein said treatment reduces ventilator use, reduces ECMO use, reduces time in the ICU or CCU, reduces hospitalization time, reduces all-cause mortality, or reduces supplemental oxygen consumption compared to a control group or historical controls. The disclosure provides a treatment method wherein said treatment reduces pulmonary exudate levels or reduces supplemental oxygen consumption compared to a baseline measurement. The disclosure provides a treatment method wherein said treatment increases subject's survival compared to a control group or historical controls. The disclosure provides a treatment method wherein the viral infection is a coronavirus selected from the group consisting of SARS-CoV, HCoV NL63, HKUl, MERS-CoV, and SARS-CoV-2. The disclosure provides a treatment method wherein the coronavirus is SARS-CoV-2. The disclosure provides a treatment method for improving, compared to a baseline measurement, the blood oxygenation level of a subject diagnosed with a viral infection, the treatment method comprising: selecting a subject diagnosed with a viral infection and in need of improving, compared to a baseline measurement, blood oxygenation level; administering to the patient at least one agent which depletes guanosine-containing nucleosides and nucleotides; and prior to, concurrently with, or subsequently to step (ii), administering to the patient a diet which depletes guanosine-containing nucleosides and nucleotides. thereby improving the subject's blood oxygenation level wherein the subject's blood oxygenation level is improved compared to a baseline measurement. The disclosure provides a treatment method wherein the at least one agent which depletes guanosine-containing nucleosides and nucleotides is selected from the group consisting of mycophenolate, IMPDH enzyme inhibitors, agents which bind to guanine, or guanosine-containing nucleosides and nucleotides, and combinations thereof. The disclosure provides a treatment method wherein the diet which depletes guanosine-containing nucleosides and nucleotides contains a nucleotide content which is selected from the group consisting of about 1000 mg/day, of about 750 mg/day, of about 500 mg/day, of about 250 mg/day, of about 100 mg/day, of about 75 mg/day, of about 50 mg/day, and of about 25 mg/day of nucleotide. The disclosure provide a treatment method wherein the baseline measurement is determined prior to infection, early in the subject's infection, from a standard, compared to a control group, or an historical control value. The disclosure provides a treatment method wherein the subject is further diagnosed with acute respiratory distress syndrome (ARDS). The disclosure provides a treatment method wherein the blood oxygenation level is determined using the oxygenation index, the oxygenation saturation index, pulse oximetry, and combinations thereof. The disclosure provides a treatment method wherein the blood oxygenation level is determined at a time point selected from the group consisting of one day, two days, three days, four days, five days, six days, seven days, eight days, nine days, 10 days, 11 days, 12 days, 13 days, two weeks, three weeks, one month, two months, and three months. The disclosure provides a treatment method wherein said treatment reduces ventilator use, reduces ECMO use, reduces time in the ICU or CCU, reduces hospitalization time, reduces all-cause mortality, or reduces supplemental oxygen consumption compared to a control group or historical controls.

The disclosure provides a treatment method wherein said treatment reduces pulmonary exudate levels or reduces supplemental oxygen consumption compared to a baseline measurement. The disclosure provides a treatment method wherein said treatment increases subject's survival compared to a control group or historical controls. The disclosure provide a treatment method wherein the viral infection is a coronavirus selected from the group consisting of SARS-CoV, HCoV NL63, HKUl, MERS-CoV, and SARS-CoV-2. The disclosure provides a treatment method wherein the coronavirus is SARS-CoV-2.

The disclosure provides a treatment method for improving, compared to a baseline measurement, the blood oxygenation level of a subject diagnosed with a viral infection, the treatment method comprising: selecting a subject diagnosed with a viral infection and in need of improving, compared to a baseline measurement, blood oxygenation level; administering to the patient at least one first agent which depletes guanosine-containing nucleosides and nucleotides; and administering to the patient at least one second agent, wherein the subject's blood oxygenation level is improved compared to a baseline measurement. The disclosure provides a treatment method wherein the at least one agent which depletes guanosine-containing nucleosides and nucleotides is selected from the group consisting of mycophenolate, IMPDH enzyme inhibitors, agents which bind to guanine, or guanosine-containing nucleosides and nucleotides, and combinations thereof. The disclosure provides a treatment method wherein the at least one second agent which is selected from the group consisting of minocycline, doxycycline, tetracycline, and tetracycline derivatives and combinations thereof. The disclosure provides a treatment method wherein the at least one first agent and at least one second agent are administered concurrently. The disclosure provides a treatment method wherein the at least one first agent and at least one second agent are administered in the same dosage form. The disclosure provides a treatment method wherein the at least one first agent and at least one second agent are administered in separate dosage forms. The disclosure provides a treatment method wherein the baseline measurement is determined prior to infection, early in the subject's infection, from a standard, compared to a control group, or an historical control value. The disclosure provides a treatment method wherein the subject is further diagnosed with acute respiratory distress syndrome (ARDS). The disclosure provides a treatment method wherein the blood oxygenation level is determined using the oxygenation index, the oxygenation saturation index, pulse oximetry, and combinations thereof. The disclosure provides a treatment method wherein the blood oxygenation level is determined at a time point selected from the group consisting of one day, two days, three days, four days, five days, six days, seven days, eight days, nine days, 10 days, 11 days, 12 days, 13 days, two weeks, three weeks, one month, two months, and three months. The disclosure provides a treatment method wherein said treatment reduces ventilator use, reduces ECMO use, reduces time in the ICU or CCU, reduces hospitalization time, reduces all-cause mortality, or reduces supplemental oxygen consumption compared to a control group or historical controls. The disclosure provides a treatment method wherein said treatment reduces pulmonary exudate levels or reduces supplemental oxygen consumption compared to a baseline measurement. The disclosure provides a treatment method wherein said treatment increases subject's survival compared to a control group or historical controls. The disclosure provides a treatment method wherein the viral infection is a coronavirus selected from the group consisting of SARS-CoV, HCoV NL63, HKUl, MERS-CoV, and SARS-CoV-2. The disclosure provides a treatment method wherein the coronavirus is SARS-CoV-2.

The disclosure provides a treatment method for improving, compared to a baseline measurement, the blood oxygenation level of a subject diagnosed with a viral infection, the treatment method comprising: (i) selecting a subject diagnosed with a viral infection and in need of improving, compared to a baseline measurement, blood oxygenation level; (ii) administering to the patient at least one first agent which depletes guanosine-containing nucleosides and nucleotides; and (iii) administering to the patient at least one second agent, (iv) prior to, concurrently with, or subsequently to steps (ii) or (iii), administering to the patient a diet which depletes guanosine-containing nucleosides and nucleotides; and wherein the subject's blood oxygenation level is improved compared to a baseline measurement. The disclosure provides a treatment method wherein the at least one agent which depletes guanosine-containing nucleosides and nucleotides is selected from the group consisting of mycophenolate, IMPDH enzyme inhibitors, agents which bind to guanine, or guanosine-containing nucleosides and nucleotides, and combinations thereof. The disclosure provides a treatment method wherein the at least one second agent which is selected from the group consisting of minocycline, doxycycline, tetracycline, and tetracycline derivatives and combinations thereof. The disclosure provides a treatment method wherein the diet which depletes guanosine-containing nucleosides and nucleotides contains a nucleotide content which is selected from the group consisting of about 1000 mg/day, of about 750 mg/day, of about 500 mg/day, of about 250 mg/day, of about 100 mg/day, of about 75 mg/day, of about 50 mg/day, and of about 25 mg/day of nucleotide. The disclosure provides a treatment method wherein the at least one first agent and at least one second agent are administered concurrently. The disclosure provides a treatment method wherein the at least one first agent and at least one second agent are administered in the same dosage form. The disclosure provides a treatment method wherein the at least one first agent and at least one second agent are administered in separate dosage forms. The disclosure provides a treatment method wherein the baseline measurement is determined prior to infection, early in the subject's infection, from a standard, compared to a control group, or an historical control value. The disclosure provides a treatment method wherein the subject is further diagnosed with acute respiratory distress syndrome (ARDS). The disclosure provides a treatment method wherein the blood oxygenation level is determined using the oxygenation index, the oxygenation saturation index, pulse oximetry, and combinations thereof. The disclosure provides a treatment method wherein the blood oxygenation level is determined at a time point selected from the group consisting of one day, two days, three days, four days, five days, six days, seven days, eight days, nine days, 10 days, 11 days, 12 days, 13 days, two weeks, three weeks, one month, two months, and three months. The disclosure provides a treatment method wherein said treatment reduces ventilator use, reduces ECMO use, reduces time in the ICU or CCU, reduces hospitalization time, reduces all-cause mortality, or reduces supplemental oxygen consumption compared to a control group or historical controls. The disclosure provides a treatment method wherein said treatment reduces pulmonary exudate levels or reduces supplemental oxygen consumption compared to a baseline measurement. The disclosure provides a treatment method wherein said treatment increases subject's survival compared to a control group or historical controls. The disclosure provides a treatment method wherein the viral infection is a coronavirus selected from the group consisting of SARS-CoV, HCoV NL63, HKUL, MERS-CoV, and SARS-CoV-2. The disclosure provides a treatment method wherein the coronavirus is SARS-CoV-2. The disclosure provides a treatment method for improving, compared to a baseline measurement, the symptoms of a subject diagnosed with a viral infection, the treatment method comprising selecting a subject diagnosed with a viral infection and in need of improving, compared to a baseline measurement, blood oxygenation level and administering a treatment selected from the group consisting of: (i) administering to the patient at least one first agent which depletes guanosine-containing nucleosides and nucleotides; and (ii) administering to the patient at least one second agent, (iii) prior to, concurrently with, or subsequently to steps (ii) or (iii), administering to the patient a diet which depletes guanosine-containing nucleosides and nucleotides; and (iv) combinations thereof, wherein the subject's symptoms are improved compared to a baseline measurement. The disclosure provides a treatment method wherein the symptoms include fever, cough, sore throat, malaise, headache, muscle pain, nausea, vomiting, diarrhea, and loss of taste or smell, without shortness of breath or dyspnea, as respiratory rate 20 breaths per minute, heart rate 90 beats per minute; with saturation of oxygen (SpO2)>93% on room air at sea level, moderate illness or shortness of breath at rest, or respiratory distress, respiratory rate 30 per minute, heart rate 125 per minute, SpO2:′·S 93% on room air at sea level or PaO2/FiO2<300, Respiratory failure, or combinations thereof. The disclosure provides a treatment method wherein the at least one agent which depletes guanosine-containing nucleosides and nucleotides is selected from the group consisting of mycophenolate, IMPDH enzyme inhibitors, agents which bind to guanine, or guanosine-containing nucleosides and nucleotides, and combinations thereof. The disclosure provides a treatment method wherein the at least one second agent which is selected from the group consisting of minocycline, doxycycline, tetracycline, and tetracycline derivatives and combinations thereof. The disclosure provides a treatment method wherein the diet which depletes guanosine-containing nucleosides and nucleotides contains a nucleotide content which is selected from the group consisting of about 1000 mg/day, of about 750 mg/day, of about 500 mg/day, of about 250 mg/day, of about 100 mg/day, of about 75 mg/day, of about 50 mg/day, and of about 25 mg/day of nucleotide. The disclosure provides a treatment method wherein the at least one first agent and at least one second agent are administered concurrently. The disclosure provides a treatment method wherein the at least one first agent and at least one second agent are administered in the same dosage form. The disclosure provides a treatment method wherein the at least one first agent and at least one second agent are administered in separate dosage forms. The disclosure provides a treatment method wherein the baseline measurement is determined prior to infection, early in the subject's infection, from a standard, compared to a control group, or an historical control value. The disclosure provides a treatment method wherein the subject is further diagnosed with acute respiratory distress syndrome (ARDS). The disclosure provides a treatment method wherein the blood oxygenation level is determined using the oxygenation index, the oxygenation saturation index, pulse oximetry, and combinations thereof. The disclosure provides a treatment method wherein the blood oxygenation level is determined at a time point selected from the group consisting of one day, two days, three days, four days, five days, six days, seven days, eight days, nine days, 10 days, 11 days, 12 days, 13 days, two weeks, three weeks, one month, two months, and three months. The disclosure provides a treatment method wherein said treatment reduces ventilator use, reduces ECMO use, reduces time in the ICU or CCU, reduces hospitalization time, reduces all-cause mortality, or reduces supplemental oxygen consumption compared to a control group or historical controls. The disclosure provides a treatment method wherein said treatment reduces pulmonary exudate levels or reduces supplemental oxygen consumption compared to a baseline measurement. The disclosure provides a treatment method wherein said treatment increases subject's survival compared to a control group or historical controls. The disclosure provides a treatment method wherein the viral infection is a coronavirus selected from the group consisting of SARS-CoV, HCoV NL63, HKUl, MERS-CoV, and SARS-CoV-2. The disclosure provides a treatment method wherein the coronavirus is SARS-CoV-2.

The disclosure provides a pharmaceutical composition comprising: the at least one agent which depletes guanosine-containing nucleosides and nucleotides selected from the group consisting of mycophenolate, IMPDH enzyme inhibitors, agents which bind to guanine, or guanosine-containing nucleosides and nucleotides, and combinations thereof; and at least one agent selected from the group consisting of minocycline, doxycycline, tetracycline, tetracycline derivatives, and combinations thereof, wherein the pharmaceutical composition further comprises at least one pharmaceutically acceptable excipient. The disclosure provides a pharmaceutical composition wherein the pharmaceutical composition is formulated or manufactured as a liquid, an elixir, an aerosol, a spray, a powder, a tablet, a pill, a capsule, a gel, a geltab, a nanosuspension, a nanoparticle, an extended release dosage form, or a topical formulation. The disclosure provides a pharmaceutical composition wherein the pharmaceutical composition is formulated or manufactured as a liquid, an elixir, an aerosol, a spray, a powder, a tablet, a pill, a capsule, a gel, a geltab, a nanosuspension, a nanoparticle, an extended release dosage form, or a topical formulation. The disclosure provides a pharmaceutical composition wherein the pharmaceutical composition is in a form for topical administration. The disclosure provides a pharmaceutical composition wherein the at least one first agent and at least one second agent are in the same dosage form. The disclosure provides a pharmaceutical composition wherein the at least one first agent and at least one second agent are in separate dosage forms. The disclosure provides a pharmaceutical composition wherein the pharmaceutical composition is in a form for topical administration.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### BRIEF DESCRIPTION OF THE DRAWINGS

The invention will be described in conjunction with the following drawings in which like reference numerals designate like elements and wherein:

FIG. 1 is a chart showing fever course in a patient with markers of each treatment initiation. (Note: 98.6° F.=normal=37° C.).

FIG. 2 is a chart showing Bivariate Fit of Temperature by Day Number for the patient of Example 6.

FIG. 3 is a chart showing Bivariate Fit of Cough by Day Number for the patient of Example 6.

FIG. 4 is a chart showing Bivariate Fit of Sneezing/runny nose By Day Number for the patient of Example 6.

### DETAILED DESCRIPTION

As used herein the term “active pharmaceutical ingredient” (“API”) or “pharmaceutically active agent” is a drug or agent which can be employed as disclosed herein and is intended to be used in the human or animal body in order to heal, to alleviate, to prevent or to diagnose diseases, ailments, physical damage or pathological symptoms; allow the state, the condition or the functions of the body or mental states to be identified; to replace active substances produced by the human or animal body, or body fluids; to defend against, to eliminate or to render innocuous pathogens, parasites or exogenous substances or to influence the state, the condition or the functions of the body or mental states. Drugs in use can be found in reference works such as, for example, the Rote Liste or the Merck Index. Examples which may be mentioned include, for example, tretinoin.

As used herein, “pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of the active agent. The pharmaceutically acceptable salts include the conventional non-toxic salts, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and other known to those of ordinary skill in the pharmaceutical sciences. Lists of suitable salts are found in texts such as Remington's Pharmaceutical Sciences, 18th Ed. (Alfonso R. Gennaro, ed.; Mack Publishing Company, Easton, Pa., 1990); Remington: the Science and Practice of Pharmacy 19*^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Oilman's: the Pharmacological Basis of Therapeutics (Louis S. Goodman and Lee E. Limbird, eds.; McGraw Hill, 1992), the disclosures of which are hereby incorporated by reference.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

As used herein, the term “acute respiratory distress syndrome” or “ARDS” describes an acute inflammatory syndrome featuring diffuse pulmonary edema and ultimately respiratory failure. The clinicopathological aspects include severe inflammatory injury to the alveolar-capillary barrier, surfactant depletion, and loss of aerated lung tissue. Under the most recent definition, the Berlin definition (Ranieri V M, et al. Acute respiratory distress syndrome: the Berlin Definition. JAMA. 2012; 307(23):2526-33), ARDS is defined by the presence within 7 days of a known clinical insult or new or worsening respiratory symptoms of a combination of acute hypoxemia (PaO2/FiO2:′S300 mmHg), in a ventilated patient with a positive end-expiratory pressure (PEEP) of at least 5 cmH2O, and bilateral opacities not fully explained by heart failure or volume overload. The Berlin definition uses the PaO2/FiO2 ratio to distinguish mild ARDS (200<PaO2/FiO2:S300 mmHg), moderate ARDS (100<PaO2/FiO2:S200 mmHg), and severe ARDS (PaO2/FiO2:Sl 00 mmHg).

The most widely used means of quantifying ARDS severity relies on a four-point lung injury scoring system (Murray Score or LIS). It is based on the level of PEEP. the ratio of the partial pressure of arterial oxygen (PaO2) to the fraction of inspired oxygen (FiO2), the dynamic lung compliance and the degree of radiographic infiltration of pulmonary exudate (Ashbaugh D, et al. Acute respiratory distress in adults. The Lancet. 1967; 290:319-23). Although the LIS has been widely used in clinical studies and a score of >3.0 is commonly used as a qualifying threshold for support with extracorporeal membrane oxygenation (ECMO), it cannot predict outcome during the first 24-72 hours of ARDS (Bernard G R, et al. The American-European consensus Conference on ARDS. definitions, mechanisms, relevant outcomes, and clinical trial coordination. Am J Respir Crit Care Med 1994; 149:818-24). When the scoring system is used 4-7 days after the onset of the syndrome, scores of 2.5 or higher predicted a complicated course requiring prolonged mechanical ventilation (Ferguson N D, et al. The Berlin definition of ARDS: an expanded rationale, justification, and supplementary material. Intensive Care Med 2012; 38:1573-82).

ARDS results from acute inflammation affecting the lung's gas exchange surface, the alveolar-capillary membrane (Fanelli V. Ranieri V M. Mechanisms and clinical consequences of acute lung injury. Ann Am Thorac Soc. 2015: 12(Supplement 1):S3-S8). The acute inflammation produces high permeability pulmonary edema with associated recruitment of neutrophils and other mediators of inflammation. The resulting acute inflammatory exudate inactivates surfactant leading to collapse and consolidation of distal airspaces with progressive loss of the lung's gas exchange surface area. This would be compensated for by hypoxic pulmonary vasoconstriction, if the inflammatory process did not also effectively paralyse the lung's means of controlling vascular tone, thereby allowing deoxygenated blood to cross unventilated lung units on its way to the left heart. The combination of these two processes causes profound hypoxemia and eventually type 2 respiratory failure as hyperventilation fails to keep pace with carbon dioxide production (Griffiths M J D, et al. Guidelines on the management ofacute respiratory distress syndrome BMJ Open Respiratory Research 2019; 6:e000420. doi: 10.1136/bmjresp-2019-000420).

As used herein, the term “multi-particulates” refers to one or more unit dosage systems such as, but not limited to, pellets, beads, spheres, mini-tablets, seeds, spheroids or granules with modified drug release profile. The multi-particulates comprise a drug-release controlling and/or drug-protecting film or matrix, such as a polymeric film or matrix, whose intactness or efficiency is susceptible to certain conditions such as heat or mechanical forces that may occur during post-processing. The expression “core material” describes the nature of the interior part of multi-particulates that may also comprise a functional coat. Exemplary “core-materials” may be pellets (spherical matrix systems that contain a drug and further excipients), granules (less spherical particles that are almost entirely composed of drug) or nonpareils (spherical particles without drug).

As used herein, the term “about” when used in conjunction with a stated numerical value or range has the meaning reasonably ascribed to it by a person skilled in the art, i.e., denoting somewhat more or somewhat less than the stated value or range.

### Method for Depletion of Guanosine-Containing Nucleosides and Nucleotides.

Guanine is a purine base which is a component of nucleosides such as guanosine and nucleotides such as guanosine monophosphate, guanosine diphosphate, and guanosine triphosphate, cyclic guanosine monophosphate, etc. Guanine is required for replication of most DNA and RNA. It is also a component of the ubiquitous G protein receptors, which are involved in innumerable signaling pathways.

Most microorganisms that cause disease do so partially by replicating themselves in high numbers. This includes viruses, bacteria, fungi, and parasites. This requires replication of DNA in bacteria, fungi, and parasites, and replication of DNA or RNA in viruses.

It has been demonstrated that restriction or complete inhibition of guanine supplies prevents replication of some viruses and other pathogenic organisms such as bacteria. This can be accomplished by several chemical agents (e.g. mycophenolic acid, tiazofurin, selenazofurin, ribavirin, 5-ethinyl-1-D-ribofuranosylimidazole-4-carboxamide))(Neyts, J., and De Clercq, E. “Mycophenolate Mofetil Strongly Potentiates the Anti-herpesvirus Activity of Acyclovir.” Antiviral research 40 (1998): 53-56). Restriction of guanine supplies is generally not used in medical treatment of any infectious disease. It should be noted that early studies of mycophenolate showed relatively rapid emergence of bacterial resistance, and perhaps this is the reason why this strategy has not been adopted. Additionally, it is possible to significantly deplete a human's supply of guanosine by a novel selective diet restriction which is described below.

### Part A—Methods for Depletion of Guanosine-Containing Nucleosides and Nucleotides: Specialized Diets and Dietary Program

Part A can be implemented using either or both of the following approaches.

Part A1—Administering agents which depletes guanosine-containing nucleosides and nucleotides.

One approach is that of administering agents (e.g., chemicals, or molecules such as immunoglobulins) that have the effect of reducing the content of guanosine-containing nucleosides and nucleotides in particular tissues or the whole body. One way to do this, for example, is that used by mycophenolate and similar compounds that inhibit the IMPDH enzyme, which is necessary for the production of guanosine monophosphate, a key intermediate in the nucleotide synthesis pathway. Another approach would be to administer agents which bind to guanine, or guanosine-containing nucleosides and nucleotides, to reduce their availability.

Part A2—Dietary restriction of guanosine intake and substances used by the body to synthesize guanosine. Describe below are specialized diets that have been constructed and used which specifically reduce the dietary intake of guanosine.

It utilizes diets which are low in nucleic acids and their components, but which are not nucleotide-free. The diets contain approximately 3% to 50% of the amount by weight of nucleotides seen in the normal western diet (2000 mg/day, from Ekelman, K Disodium 5′Guanylate and Disodium 5′-Inosate. WHO Food Additives Series, No. 32 (1993), and preferably 10%-40%. The percentage of nucleotides, nucleosides, and other nucleic acid components in specific foods has been published by different researchers using various analytic techniques over the years (e.g. Lassek, E, and A Montag. “Nucleic Acid Components in Carbohydrate-rich Food.” Zeitschrift fur Lebensmittel-Untersuchung und-Forschung 190, no. 1 (1990): doi:1689090; Souci, SW, W Fachmann, H Kraut, Eva Kirchhoff, and Forschungsanstalt Forschungsanstalt ftir Deutsche. Food Composition and Nutrition Tables. Stuttgart: Medpharm, 2008; Brule, D, G Sarwar, and L Savoiet. “Purine Content of Selected Canadian Food Products.” Journal of Food Composition and Analysis 1, no. 2 (1988): 130-138). A survey of the world literature on nucleic acid content of foods was conducted. In some cases where the individual nucleotides were not reported, the amount of total nucleotides and of guanosine-containing nucleotides could be estimated from the reported purine content. A set of diets with different percentages of nucleotides (compared to the typical Western diet) was created. These range between about 10% to 40% of the typical Western diet. A registered dietitian created these diets which were low in nucleotides but balanced for other necessary nutrients.

Choosing a diet with a given percent (or range) of nucleotides is optimized by medical evaluation of the condition and needs of the individual patient. Medical evaluation may include the following: level of antibody titers such as those of anti-nuclear antibody, anti-dsDNA antibodies, anti-guanosine antibody, evaluation of the presence and degree of organ damage in kidneys, lungs, joints, brain, and skin, subjective symptomatology such as pain, headaches, and evaluation criteria.

Additionally, it is proposed that all patients with pathogenic infections, and individuals susceptible to developing pathogenic infections, will benefit from diets in which the nucleotide level is 45% or less than the typical western diet, and in which the guanosine and guanosine-containing nucleotides are less than 45% of the typical western diet.

2. The diets initially recommended by the physician to the patient are low in their content of guanine. a nitrogenous base. and/or guanosine. a nucleoside. compared to the usual Western d. The patient's response to the diet is evaluated over time and diets with sequentially more nucleotide content and more palatability are recommended so that the compliance and tolerability and palatability is at such a level that the patient can for years be maintained on a reduced nucleotide diet. For instance, if the patient is started by the physician on a Step 1 diet, it is likely that after a period of weeks or months the patient will be advanced to a Step 2 diet which has a greater nucleotide content, reflected in a larger range of foods which can be taken, and is therefore more palatable.

(Nucleotide-free diets are described in: Rudolph, F B, A D Kulkarni, WC Fanslow, R P Pizzini, S Kumar, and CT Van Buren. “Role of RNA As a Dietary Source of Pyrimidines and Purines in Immune Function.” Nutrition 6, no. 1 (1990): 45-52; Kulkarni, AD, F B Rudolph, and C T Van Buren. “The Role of Dietary Sources of Nucleotides in Immune Function: A Review.” The Journal of nutrition 124, no. 8 Suppl (1994): 1442S-1446S.: 45-52).

The inventor's reduced nucleotide diets are fundamentally different from nucleotide-free diets. The nucleotide-free diets used in published animal experiments were almost entirely devoid of nucleotides and were said to contain a level of only 0.001% (Rudolph, F B, AD Kulkarni, W C Fanslow, RP Pizzini, S Kumar, and C T Van Buren. “Role of RNA As a Dietary Source of Pyrimidines and Purines in Immune Function.” Nutrition 6, no. 1 (1990): 45-52).

The inventor has conducted extensive analysis of the nucleotide content of human foods from a variety of sources and evaluated the nutritional content and palatability of potential nucleotide-free diets. The inventor has concluded that it is not practical for most people to stay on a nucleotide-free diet in a compliant manner for the period of months required to obtain substantial clinical benefit from this approach. A nucleotide-free diet is unlikely to be sufficiently palatable for extended use and would deter compliance. Also, use of a nucleotide-free diet for months in humans would likely lead to other dietary deficiencies.

As set forth above, the disclosure provides treatment of a patient with a diet which contains approximately 3% to 50% of the amount by weight of nucleotides seen in the normal western diet, which contains about 2000 mg/day of nucleotides. In exemplary embodiments, the diet of the disclosure contains a nucleotide content of about 1000 mg/day, of about 750 mg/day, of about 500 mg/day, of about 250 mg/day, of about 100 mg/day, of about 75 mg/day, of about 50 mg/day, of about 25 mg/day. In exemplary embodiments, the diet of the disclosure contains a nucleotide content compared to the normal Western diet of about 50%, of about 40%, of about 30%, of about 20%, of about 10%, of about 5%, of about 3%. In exemplary embodiments, the diet of the disclosure contains a nucleotide content compared to the normal Western diet of 3-50%, of about 10-40%, of about 20-30%, of about 3-40%, of about 3-30%, of about 10-30%, of about 10-20%.

2. Example diets: The nucleotide content of one Example Diet is about 28f5% of the typical Western diet. The nucleotide content of another Example Diet is about 43±5% of the typical Western diet.

3. The diet program is comprised of a period of time, for example, days, weeks, or months, e.g., 6 months, during which a physician and a dietician evaluate and work to optimize diet treatment for each pathogenic infection patient.

a. The physician initially evaluates the severity of the patient's disease based on signs and symptoms, laboratory tests, evidence of organ damage, etc., and then recommends a specific diet. The patient is followed over the next few months with repeat followup disease evaluations and diet adjustments by the physician. The patient may be asked to keep, as individually necessary, logs of symptoms such as headaches, skin rashes, joint pains, etc. These logs are periodically reviewed by the physician.

b. The dietician has an initial meeting with the patient in which the practical aspects of the diet program are explained in detail. This first visit includes describing weighing the foods to be included in the diet, characterizing the portion size for each type of food, keeping a food log, advising on the effect of cooking on food nucleotide content, etc. The patient will have one or more follow-up visits, as well as occasional other phone, email or other communications with the patient to answer questions and direct the treatment.

c. Depending on the severity of the patient's disease, an initial diet will be selected. As the patient stabilizes clinically or based on laboratory testing, the patient will be moved through a series of diets with increasing nucleotide content, but which are still substantially lower compared to the typical Western diet. It is believed that for each patient there is a threshold for reactivity, and that if the diets are below that threshold the patient will have successfully minimized signs, symptoms, and progression of the pathogenic infection. The goal of the program is then to put the patient on a convenient and palatable maintenance diet which they can pragmatically follow for a period of years.

d. A specialized version of the low-guanosine diet has been constructed which meets the needs of pregnant women.

### Mycophenolate

As used herein, “mycophenolates” refers herein to mycophenolic acid (“MPA”) and its analogs, and their pharmaceutically acceptable salts, derivatives, prodrugs, and metabolites. Exemplary mycophenolates for use in the present disclosure include mycophenolic acid and mycophenylate mofetil. Mycophenolic acid, or 6-(4-hydroxy-6-methoxy-7-methyl-3-oxo-1,3-dihydroisobenzofuran-5-yl)-4-methyl-hex-4-enoic acid, has the structure

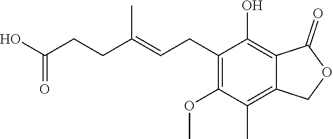 [SMILES: COc1c(C)c2c(c(O)c1C/C=C(\C)CCC(=O)O)C(=O)OC2]

Mycophenolate mofetil is the 2-morpholinoethyl ester of mycophenolic acid, and has the formula:

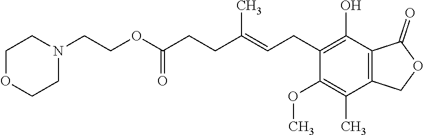 [SMILES: COc1c(C)c2c(c(O)c1C/C=C(\C)CCC(=O)OCCN1CCOCC1)C(=O)OC2]

Analogs of mycophenolic acid that have high IMPDH-inhibiting activity are also useful in the practice of the present disclosure include compounds with varying substituents in the 2-, 4-, 5-, and 6-positions on the mycophenolate core structure, as well as pharmaceutically acceptable salts, derivatives, prodrugs, and metabolites of such mycophenolate analogs. Such compounds are described for example, in the following U.S. patents incorporated herein by reference: 5,688,529 Mycophenolate mofetil high dose oral suspensions; 5,633,279 5-Substituted derivatives of mycophenolic acid 5,554,612 4-Amino-6-substituted mycophenolic acid and derivatives 5,538,969 4-Amino derivatives of 5-substituted mycophenolic acid 5,536,747 6-Substituted mycophenolic acid and derivatives 5,493,030 5-Substituted derivatives of mycophenolic acid; 5,444,072 6-Substituted mycophenolic acid and derivatives 5,441,953 4-Amino derivatives of mycophenolic acid 5,380,879 Derivatives of mycophenolic acid 4,861,776 Heterocyclic aminoalkyl esters of mycophenolic acid and derivatives thereof; 4,753,935 Morpholinoethylesters of mycophenolic acid; 4,748,173 Heterocyclic aminoalkyl esters of mycophenolic acid and derivatives thereof, 4,727,069 Heterocyclic aminoalkyl esters of mycophenolic acid, derivatives thereof

The art teaches that dose and administration time of mycophenolic acid for antiviral effects are surprisingly different than the current dose and administration time for prevention of transplant rejection. It is important to understand that mycophenolic acid's anti-viral effect is likely to require a small fraction (about 1-2% or less) of the clinical dose which is approved and commonly used for prevention of transplant rejection. For instance, Chan et al., 2013, p. 612 indicated that for a virus in which mycophenolic acid demonstrated an EC so of 0.17 mcg/ml, the usual clinical doses of mycophenolic acid would result in local concentrations 60-300× greater than needed. This is directly comparable for our results. For instance, in one of the Norovirus tests we document here, ECso of 0.151 mcg/mL was reported. Even less mycophenolic acid would be needed to treat viruses such as Influenza A HNI and RSV in which we report ECso <0.10 mcg/ml. Dramatically lower doses may suffice for mycophenolic acid. Therefore, it is not only possible to achieve adequate body levels of mycophenolic acid to treat these viruses clinically, but also a small fraction of the current clinical dose may be more than adequate. To et al, 2016 also presented data on page 1812 supporting these conclusions.

Mycophenolic acid, when currently used clinically to prevent transplant rejection in an individual patient, typically is given for months or years at doses of 2000 mg to 3000 mg per day, to block lymphocyte cell proliferation. In contrast, the highly effective treatments of the disclosure may require, for example, just one to two weeks of administration in an individual patient.

A third beneficial point is that the combination of low dose and short time of administration of mycophenolic acid to achieve the pharmaceutical effect, is likely to drastically decrease the occurrence of those side effects (adverse events) which have typically been reported clinically where it is currently used at very high dose for very long periods of time. At low dose for short periods of time it is extremely unlikely to have immunosuppressive effects which could be detrimental to the body's resistance to other pathogens.

A fourth beneficial point is that the low dose, short time of administration usage described above may also serve to reduce the teratogenic potential of this drug, which occurs when it is used at high dose for long periods of time.

A fifth beneficial point regarding dosing and time of administration relates to those pathogenic infections which result in development of autoimmunity causing neurological illness such as Guillain-Barre Syndrome, encephalitis, myelitis, paralysis, confusion, weakness, etc. In those situations, for instance, the low effective dose can be titrated up so that an appropriate degree of mild immunosuppression is achieved simultaneously or shortly after the effect to block the autoimmunity from causing neurological illness. (It has been demonstrated that the immunosuppressive effect of mycophenolic acid on lymphocytes is dose-dependent, with lower doses having less effect (Vethe et al., 2008).) For example, West Nile infection is well-documented to cause neurological illness. In a mouse model where West Nile infection leads to neurological illness, administration of appropriate doses of mycophenolic acid is demonstrated to mitigate the degree and occurrence of neurological illness.

The following references are incorporated herein in their entirety: Chan, J. F., Chan, K. H., Kao, R Y., To, K. K., Zheng, B. J., Li, C. P., Li, P. T., Dai, J., Mok, F. K., Chen, H., Hayden, F. G., Yuen, K. Y. (2013). Broad-spectrum antivirals for the emerging Middle East respiratory syndrome coronavirus. J Infect 67: 606-616. To, K. K., Mok, K. Y., Chan, A. S., Cheung, N. N., Wang, P., Lui, Y. M., Chan, J. F., Chen, H., Chan, K. H., Kao, R. Y., Yuen, K. Y. (2016). Mycophenolic acid, an immunomodulator, has potent and broad-spectrum in vitro antiviral activity against pandemic, seasonal and avian influenza viruses affecting humans. J Gen Virol 97: 1807-1817. Vethe, N. T., Bremer, S., Rootwelt, H., Bergan, S. (2008). Pharmacodynamics of mycophenolic acid in CD4+ cells: a single-dose study of IMPDH and purine nucleotide responses in healthy individuals. Therapeutic drug monitoring 30: 647-655.

### Effectiveness of Mycophenolic Acid Against Influenza

1. Data supporting the effectiveness of mycophenolic acid in treating important and currently clinically relevant strains of influenza.

To et al. (2016) pointed out that the demonstration by Chan et al. (2013) of effectiveness of mycophenolic acid against the historical Influenza A(H1N1) strain A/WSN/1933 in a chemical screening assay, was insufficient to determine whether it would be effective against other strains of influenza. To et al. (2016) then experimentally showed that mycophenolic acid is effective against pandemic flu H/415, which is A/Hong Kong/415742/2009, and is sometimes written as A(HINI)pdm09. They also showed it to be effective against seasonal influenza A(H3N2) virus, and avian-origin influenza A (H7N9), as well as other influenza A and influenza B viruses. These strains which they tested include those which are currently clinically relevant.

2. The disclosure provides for the use of mycophenolic acid to be active also against the following respiratory viruses: coronaviruses, such as SARS-Cov2, Influenza A H3N2; Influenza A H5N1 (low path); Influenza B (Victoria); Influenza B (Yamagata); Parainfluenza virus-3; Rhinovirus-14; Influenza A H7N9 virus; Influenza A H5N1 (high path).

The following references are incorporated herein by reference in their entireties: Chan, J. F., Chan, K. H., Kao, R. Y., To, K. K., Zheng, B. J., Li, C. P., Li, P. T., Dai, J., Mok, F. K., Chen, H., Hayden, F. G., Yuen, K. Y. (2013). Broad-spectrum antivirals for the emerging Middle East respiratory syndrome coronavirus. J Infect 67: 606-616. To, K. K., Mok, K. Y., Chan, A. S., Cheung, N. N., Wang, P., Lui, Y. M., Chan, J. F., Chen, H., Chan, K. H., Kao, R. Y., Yuen, K. Y. (2016). Mycophenolic acid, an immunomodulator, has potent and broad-spectrum in vitro antiviral activity against pandemic, seasonal and avian influenza viruses affecting humans. J Gen Virol 97: 1807-1817. (First Published Online: 1 Aug. 2016). Minocycline, Doxycycline. and other Tetracycline derivatives

The disclosure provides the use of minocycline, doxycycline and other tetracycline derivatives, including for example tetracycline, for the prevention, treatment, and/or control of COVID-19 symptoms. Minocycline is a preferred compound because it will also cross the blood brain barrier. Without being bound by any theory, it is believed that the primary mechanism is inhibition of viral replication, but other properties including antioxidant and anti-inflammatory effects on the host cells are believed to contribute. Several tetracycline derivatives have been demonstrated to inhibit other viruses, including retroviruses and other flaviviridae (Chapagain, 2012; Dutta, 2010; Michaelis, 2007; Rothan, 2014). Chapagain, M. (2012). Minocycline Protects Mice against West nile virus (WNV)-associated severe disease 18th SNIP Scientific Conference. Journal of Neuroimmune Pharmacology 7: 5-81. Dutta, K., Anirban, B. (2011) Use of minocycline in viral infections. Indian Journal of Medical Research 133: 467. Michaelis, M., Kleinschmidt, M. C., Doerr, H. W., Cinatl, J. (2007). Minocycline inhibits West Nile virus replication and apoptosis in human neuronal cells. J Antimicrob Chemother 60: 981-986. Rothan, H. A., Mohamed, Z., Paydar, M., Rahman, N. A., Yusof, R. (2014). Inhibitory effect of doxycycline against dengue virus replication in vitro. Arch Virol 159: 711-718.

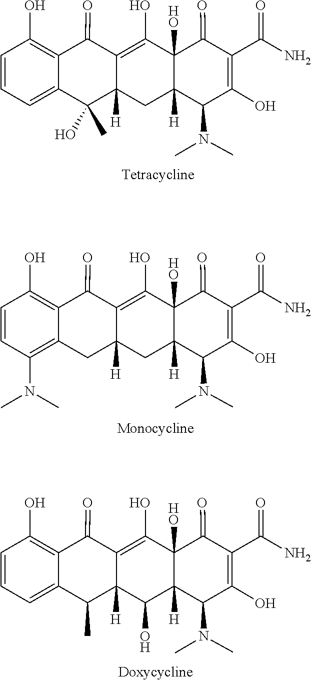 [SMILES: CN(C)[C@@H]1C(O)=C(C(N)=O)C(=O)[C@@]2(O)C(O)=C3C(=O)c4c(O)cccc4[C@@](C)(O)[C@H]3C[C@@H]12 ; CN(C)c1ccc(O)c2c1C[C@H]1C[C@H]3[C@H](N(C)C)C(O)=C(C(N)=O)C(=O)[C@@]3(O)C(O)=C1C2=O ; C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

In exemplary embodiments, formulations as disclosed herein may comprise active agent at a concentration of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, In exemplary embodiments, formulations as disclosed herein may comprise active agent at a concentration of about 1 to about 20%, of about 5% to about 25%, about 10% to about 20%, or about 15% to about 18%, about 30% to about 70%, about 35% to about 65%, about 63.13%, and about 40% to about 64% w/w.

In an exemplary formulation as disclosed herein, the active agent will represent approximately 1 wt % to 75 wt %, preferably 2 wt % to 30 wt %, more preferably 5 wt. % to 20 wt. % of the total weight.

As used herein, the term “active agent” includes, for example, mycophenolate, IMPDH enzyme inhibitors, agents which bind to guanine, or guanosine-containing nucleosides and nucleotides, minocycline, doxycycline, tetracycline, combinations thereof and derivatives of these compounds, and the free base thereof, salts thereof, isomers thereof, amorphous forms thereof, polymorphs forms thereof, coated forms thereof, crystalline forms thereof, ion par forms thereof, co crystalline forms thereof, prodrugs thereof, analogs thereof, derivatives thereof, stereoisomers forms thereof, synthetic forms thereof, alone or in combinations thereof. In certain embodiments the active agent is highly purified. In certain embodiments the active agent is a highly pure synthetic. In certain embodiments the active agent is present as a highly purified extract of active agent which comprises at least 90%, 91%, 92%, 93%, 94%, 95%, 96%, 97%, 98%, 99%, 99.5%, or 99.75% (w/w) of the formulation.

In certain embodiments the active agent is present in the formulation provided at a concentration of at least 90%, 91%, 92%, 93%, 94%, 95%, 96%, 97%, 98%, 99%, 99.5%, or 99.75%, or 100% (w/w).

In certain embodiments the active agent is 100% synthetic. In certain embodiments the active agent has a purity equal to or greater than about 90%, 91%, 92%, 93%, 94%, 95%, 96%, 97%, 98%, 99%, 99.5%, 99.75%, or 100% (w/w). In certain embodiments the active agent is produced synthetically and has a purity equal to or greater than about 90%, 91%, 92%, 93%, 94%, 95%, 96%, 97%, 98%, 99%, 99.5%, 99.75%, or 100% (w/w). In certain embodiments the active agent is a combination of active agents, and each active agent may be produced synthetically and independently have a purity equal to or greater than about 90%, 91%, 92%, 93%, 94%, 95%, 96%, 97%, 98%, 99%, 99.5%, 99.75%, or 100% (w/w).

In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, or 45 mg/kg/day. In certain embodiments, the dose of active agent is equal to or greater than, for example, about 0.001 ng/day, 0.01 ng/day, 0.025 ng/day. 0.05 ng/day, 0.1 ng/day, 0.25 ng/day, 0.5 ng/day, 1 ng/day, 10 ng/day, 25 ng/day, 50 ng/day, 100 ng/day, 250 ng/day, 500 ng/day, 1000 ng/day, 0.001 microgram/day, 0.01 microgram/day, 0.025 microgram/day, 0.050 microgram/day, 0.1 microgram/day, 0.25 microgram/day, 0.5 microgram/day, 1 microgram/day, 2.5 microgram/day, 5 microgram/day, 10 microgram/day, 25 microgram/day, 50 microgram/day, 100 microgram/day, 250 microgram/day, or 500 microgram/day. In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 ng/day. In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg/day. In exemplary embodiments, formulations of the disclosure may comprise active agent at a concentration of about 0.001 ng, 0.01 ng, 0.025 ng. 0.05 ng, 0.1 ng, 0.25 ng, 0.5 ng, 1 ng, 10 ng, 25 ng, 50 ng, 100 ng, 250 ng, 500 ng, 1000 ng, 0.001 microgram, 0.01 microgram, 0.025 microgram. 0.05 microgram, 0.1 microgram, 0.25 microgram, 0.5 microgram, 1 microgram, 2.5 microgram, 5 microgram, 10 microgram, 25 microgram, 50 microgram, 100 microgram, 250 microgram, or 500 microgram. In exemplary embodiments, formulations of the disclosure may comprise active agent at a concentration of about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 ng. In exemplary embodiments, formulations of the disclosure may comprise active agent at a concentration of about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg.

In other embodiments, the pharmaceutical compositions further comprise one or more additional materials such as a pharmaceutically compatible carrier, binder, viscosity modifier, filling agent, suspending agent, flavoring agent, sweetening agent, disintegrating agent, surfactant, preservative, lubricant, colorant, diluent, solubilizer, moistening agent, stabilizer, wetting agent, anti-adherent, parietal cell activator, anti-foaming agent, antioxidant, chelating agent, antifungal agent, antibacterial agent, or one or more combination thereof.

### Carrier

Examples of lipids that may be employed in the compositions and methods as disclosed herein include, but are not limited to, fats, oils, waxes, fatty acids, fatty acid esters, glycerides, fatty alcohols, hydrogenated vegetable oil, soybean oil, phospholipids, terpenes and the like or combinations thereof. Suitable waxes that may be employed include, but are not limited to, natural waxes, such as animal waxes, vegetable waxes, and petroleum waxes (i.e., paraffin waxes, microcrystalline waxes, petrolatum waxes, mineral waxes), and synthetic waxes. Non-limiting examples include, but are not limited to, spermaceti wax, camauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, and the like, or mixtures thereof. Mixtures of these waxes with the fatty acids may also be used. Non-limiting examples of oils that may be employed include, castor oil, soybean oil, and the like or combinations thereof. Fatty acids that may be employed in the present invention include, but are not limited to, decenoic acid, docosanoic acid, stearic acid, palmitic acid, lauric acid, myristic acid, and the like, and mixtures thereof. Suitable fatty alcohols that may be employed in the compositions as disclosed herein include, but are not limited to, cetyl alcohol, stearyl alcohol or mixtures thereof. Suitable hydrogenated vegetable oils that may be employed in the compositions as disclosed herein, include but are not limited to, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, and the like, and mixtures thereof. In one embodiment, lipids may be employed in case the carrier particles being prepared are solid lipid nanoparticles, lipid-based nanoparticles or microparticles, nanoemulsions, microemulsions, liposomes, and the like or combinations thereof

### Viral Pathogens

The disclosure provides for compositions and methods for preventing, treating, relieving, or ameliorating symptoms of viral infection, such as coronavirus infection, including SARS-CoV2 and variants thereof, including treating or preventing severe illness from coronavirus infection, such as COVID-19. Viruses which may be treated using the compositions and method as disclosed herein include DNA and RNA viruses. These include respiratory viruses such as Adenoviruses, Avian influenza, Influenza virus type A, Influenza virus type B, Measles, Parainfluenza virus, Respiratory syncytial virus (RSV), Rhinoviruses, and SARS coronavirus, gastro-enteric viruses such as Coxsackie viruses, enteroviruses such as Poliovirus and Rotavirus, hepatitis viruses such as Hepatitis B virus, Hepatitis C virus, Bovine viral diarrhea virus (surrogate), herpes viruses such as Herpes simplex 1, Herpes simplex 2, Human cytomegalovirus, and Varicella zoster virus, retroviruses such as Human immunodeficiency virus 1 (HIV-1), and Human immunodeficiency virus 2 (HW-2), as well as Dengue virus, Hantavirus, Hemorrhagic fever viruses, Lymphocytic choriomeningitis virus, Smallpox virus, Ebola virus, Rabies virus, West Nile virus (WNV) and Yellow fever virus.

Examples of viruses which may be prevented and/or treated by the compositions and methods of the disclosure include Parvoviridae; Papovaviridae (Human papilloma virus (HPV); BK polyomavirus; JC polyomavirus); Adenoviridae (Adenovirus, types 40 and 41); Herpesviridae (simplex virus type 1 (HHV-1); Herpes simplex virus type 2 (HHV-2); Macacine herpesvirus 1; Varicella-zoster virus (VZV; HHV-3); Epstein-Barr virus (EBV; HHV-4); Cytomegalovirus (CMV; HHV-5); Human Herpesvirus 6 (HHV-6); HHV-7; Kaposi's sarcoma-associated herpesvirus (HHV-8); Hepadnaviridae (Hepatitis B virus); Poxviridae (Smallpox (Variola major); Alastrim (Variola minor); Vaccinia; Cowpox; Monkeypox; Goat pox, pseudocowpox virus, bovine papular stomatitis virus, tanapox, volepox and related pox viruses such as avipox, buffalopox, racoonpox, squirrelpox, etc.); Molluscum contagiosum; Picornaviridae (Polio virus; Coxsackie A virus; Coxsackie B; virus; Foot and mouth disease; ECHO virus; Hepatitis A virus; Rhinovirus); Astroviridae; Caliciviridae (Norwalk virus; Norovirus; Sapoviruses; Hepatitis E virus); Reoviridae (Rotavirus); Togaviridae (Alpha viruses; Western equine encephalitis (WEE) virus; Eastern equine encephalitis (EEE) virus; Venezuelan equine encephalitis (VEE) virus; Chikungunya virus; Rubivirus (rubella)); Flaviviridae (Yellow fever virus; Dengue virus; St. Louis encephalitis virus; Japanese encephalitis virus; Tick-borne encephalitis virus; Omsk hemorrhagic fever virus; Al Khumra virus; Kyasanur Forest disease virus; Louping ill virus; West Nile virus; Kunjin virus; Murray Valley fever virus; Powassan virus; Hepatitis C virus; Hepatitis G virus); Coronoviridae (Respiratory illness (cold); Severe Acute Respiratory Syndrom)-corona virus (SARS-CoV)); Bunyaviridae (California encephalitis virus; La Crosse virus; Rift Valley fever virus; West Nile virus, Phleboviruses; Sandfly fever virus; Nairovirus; Hantavirus); Orthomyxoviridae (Influenza virus (types A, B & C); Paramyxoviridae (Parainfluenza virus; Respiratory syncytial virus (RSV); Hendra virus disease (formerly equine morbillivirus); Nipah virus encephalitis; Mumps Measles; Newcastle disease virus); Rhabdoviridae (Rabies virus); Filoviridae (Marburg virus (acute hemorrhagic fever); Ebola virus (acute hemorrhagic fever)); Arenaviridae (Lymphocytic choriomeningitis virus; Lassa fever virus; Lujo virus; Chapare virus; Junin virus; Machupo virus; Guanarito virus; Sabia virus); Retroviridae (Human Immunodeficiency virus (HIV) types I and II; Human T-cell leukemia virus (HLTV) type I; Human T-cell leukemia virus (HLTV) type II; Spumaviruses; Xenotropic murine leukemia virus-related (XMRV).

A non-exhaustive list of viruses and their species which can be prevented and/or treated by the compositions and methods of the invention include, for example: Abadina virus (Reoviridae), Abelson murine leukemia virus (Retroviridae), Abras virus (Bunyaviridae), Absettarov virus (Flaviviridae), Abu Hammad virus (Bunyaviridae), Abu Mina virus (Bunyaviridae), Acado virus (Reoviridae), Acara virus (Bunyaviridae), Acciptrid herpesvirus (Herpesviridae), Acheta domestica densovirus (Parvoviridae), Acrobasis zelleri entomopoxvirus (Poxviridae), Adelaide River virus (Rhabdoviridae), Adeno-associated virus (Parvoviridae), Aedes aegypti densovirus (Parvoviridae), Aedes aegypti entomopoxvirus (Poxviridae), Aedes albopictus densovirus (Parvoviridae), Aedes pseudoscutellaris densovirus (Parvoviridae), African green monkey cytomegalovirus (Herpesviridae), African green monkey HHV-like virus (Herpesviridae), African green monkey polyomavirus (Papovaviridae), African horse sickness viruses (Reoviridae), African swine fever virus, African swine fever-like viruses, AG-virus (Bunyaviridae), AG-virus, (Bunyaviridae), Agaricus bisporus virus, Aguacate virus (Bunyaviridae), Ahlum water-borne virus (Tombusviridae), Aino virus (Bunyaviridae), Akabane virus (Bunyaviridae), AKR (endogenous) murine leukemia virus (Retroviridae), Alajuela virus (Bunyaviridae), Alcelaphine herpesvirus (Herpesviridae), Alenquer virus (Bunyaviridae), Aleutian disease virus (Parvoviridae), Aleutian mink disease virus (Parvoviridae), Alfuy virus (Flaviviridae), Allerton virus (Herpesviridae), Allitrich herpesvirus (Herpesviridae), Allomyces arbuscula virus, Almeirim virus (Reoviridae), Almpiwar virus (Rhabdoviridae), Altamira virus, (Reoviridae), Amapari virus (Arenaviridae), American ground squirrel herpesvirus, (Herpesviridae), Amsacta moorei entomopoxvirus (Poxviridae), Amyelosis chronic stunt virus (Caliciviridae), Ananindeua virus (Bunyaviridae), Anatid herpesvirus (Herpesviridae), Andasibe virus (Reoviridae), Anhanga virus (Bunyaviridae), Anhembi virus (Bunyaviridae), Anomala cuprea entomopoxvirus (Poxviridae), Anopheles A virus (Bunyaviridae), Anopheles virus (Bunyaviridae), Antequera virus (Bunyaviridae), Aotine herpesvirus (Herpesviridae), Apeu virus (Bunyaviridae), Aphodius tasmaniae entomopoxvirus (Poxviridae), Apoi virus (Flaviviridae), Aransas Bay virus (Bunyaviridae), Arbia virus (Bunyaviridae), Arboledas virus (Bunyaviridae), Arbroath virus (Reoviridae), Argentine turtle herpesvirus (Herpesviridae), Arkonam virus (Reoviridae), Aroa virus (Flaviviridae), Arphia conspersa entomopoxvirus (Poxviridae), Aruac virus (Rhabdoviridae), Arumowot virus (Bunyaviridae), Asinine herpesvirus (Herpesviridae), Atlantic cod ulcus syndrome virus (Rhabodoviridae), Atlantic salmon reovirus Australia (Reoviridae), Atlantic salmon reovirus Canada (Reoviridae), Atlantic salmon reovirus USA (Reoviridae), Atropa belladonna virus (Rhabdoviridae), Aucuba bacilliform virus, Badnavirus, Aujeszky's disease virus (Herpesviridae), Aura virus (Togaviridae), Auzduk disease virus (Poxviridae), Avalon virus (Bunyaviridae), Avian adeno-associated virus (Parvoviridae), Avian carcinoma, Mill Hill virus (Retroviridae), Avian encephalomyelitis virus (Picornaviridae), Avian infectious bronchitis virus (Coronaviridae), Avian leukosis virus—RSA (Retroviridae), Avian myeloblastosis virus (Retroviridae), Avian myelocytomatosis virus (Retroviridae), Avian nephrites virus (Picomaviridae), Avian paramyxovirus (Paramyxoviridae), Avian reovirus (Reoviridae), B virus (Parvoviridae), B-lymphotropic papovavirus (Papovaviridae), Babahoya virus (Bunyaviridae), Babanki virus (Togaviridae), Baboon herpesvirus (Herpesviridae), Baboon polyomavirus (Papovaviridae), Bagaza virus (Flaviviridae), Bahia Grande virus (Rhabdoviridae), Bahig virus (Bunyaviridae), Bakau virus (Bunyaviridae), Baku virus (Reoviridae), Bald eagle herpesvirus (Herpesviridae), Bandia virus (Bunyaviridae), Bangoran virus (Rhabdoviridae), Bangui virus (Bunyaviridae), Banzi virus (Flaviviridae), Barmah Forest virus (Togaviridae), Barranqueras virus (Bunyaviridae), Barur virus (Rhabdoviridae), Batai virus (Bunyaviridae), Batarna virus (Bunyaviridae), Batken virus (Bunyaviridae), Bauline virus (Reoviridae), Beak and feather disease virus (Circoviridae), BeAn virus (Rhabdoviridae), BeAr virus (Bunyaviridae), Bebaru virus (Togaviridae), Belem virus (Bunyaviridae), Belmont virus ((Bunyaviridae)), Belterra virus (Bunyaviridae), Benevides virus (Bunyaviridae), Benfica virus (Bunyaviridae), Berne virus, (Coronaviridae), Berrimah virus (Rhabdoviridae), Bertioga virus (Bunyaviridae), Bhanj a virus (Bunyaviridae), Bimbo virus (Rhabdoviridae), Bimiti virus (Bunyaviridae), Birao virus (Bunyaviridae), BivensArm virus (Rhabdoviridae), BK virus (Papovaviridae), Bluetongue viruses (Reoviridae), Bobaya virus (Bunyaviridae), Bobia virus (Bunyaviridae), Bobwhite quail herpesvirus (Herpesviridae), Boid herpesvirus (Herpesviridae), Bombyx mori densovirus (Parvoviridae), Boolarra virus (Nodaviridae), Boraceia virus (Bunyaviridae), Border disease virus (Flaviviridae), Boma disease virus, Botambi virus (Bunyaviridae), Boteke virus, (Rhabdoviridae), Bouboui virus (Flaviviridae), Bovine adeno-associated virus (Parvoviridae), Bovine adenoviruses (Adenoviridae), Bovine astrovirus (Astroviridae), Bovine coronavirus (Coronaviridae), Bovine diarrhea virus (Flaviviridae), Bovine encephalitis herpesvirus (Herpesviridae), Bovine enteric calicivirus (Caliciviridae), Bovine enterovirus (Picornaviridae), Bovine ephemeral fever virus (Rhabdoviridae), Bovine herpesvirus (Herpesviridae), Bovine immunodeficiency virus (Retroviridae), Bovine leukemia virus (Retroviridae), Bovine mamillitis virus (Herpesviridae), Bovine papillomavirus (Papovaviridae), Bovine papular stomatitis virus (Poxviridae), Bovine parainfluenza virus (Paramyxoviridae), Bovine parvovirus (Parvoviridae), Bovine polyomavirus (Papovaviridae), Bovine Respiratory Syncytial Virus (Paramyxoviridae), Bovine rhinovirus (Picornaviridae), Bovine syncytial virus (Retroviridae), Bozo virus (Bunyaviridae), Broadhaven virus (Reoviridae), Bruconha virus (Bunyaviridae), Brus Laguna virus (Bunyaviridae), Budgerigar fledgling disease virus (Papovaviridae), Buenaventura virus (Bunyaviridae), Buffalopox virus (Poxviridae), Buggy Creek virus (Togaviridae), Bujaru virus (Bunyaviridae), Bukalasa bat virus (Flaviviridae), Bunyamwera virus (Bunyaviridae), Bunyip creek virus (Reoviridae), Bushbush virus (Bunyaviridae), Bussuquara virus (Flaviviridae), Bwamba virus (Bunyaviridae), Cache Valley virus (Bunyaviridae), Cacipacore virus (Flaviviridae), Caddo Canyon virus (Bunyaviridae), Caimito virus (Bunyaviridae), Calchaqui virus (Rhabdoviridae), California encephalitis virus (Bunyaviridae), California harbor sealpox virus (Poxviridae), Callistephus chinensis chlorosis virus (Rhabdoviridae), Callitrichine herpesvirus (Herpesviridae), Camel contagious ecthyma virus (Poxviridae), Camelpox virus (Poxviridae), Camptochironomus tentans entomopoxvirus (Poxviridae), Cananeia virus (Bunyaviridae), Canarypox virus (Poxviridae), Candiru virus (Bunyaviridae), Canid herpesvirus (Herpesviridae), Caninde virus (Reoviridae), Canine adeno-associated virus (Parvoviridae), Canine adenovirus (Adenoviridae), Canine calicivirus (Caliciviridae), Canine coronavirus (Coronaviridae), Canine distemper virus (Paramyxoviridae), Canine herpesvirus (Herpesviridae), Canine minute virus (Parvoviridae), Canine oral papillomavirus (Papovaviridae), Canine parvovirus (Parvoviridae), Canna yellow mottle virus (Badnavirus), Cape Wrath virus (Reoviridae), Capim virus (Bunyaviridae), Caprine adenovirus (Adenoviridae), Caprine arthritis encephalitis virus (Retroviridae), Caprine herpesvirus (Herpesviridae), Capuchin herpesvirus AL-(Herpesviridae), Capuchin herpesvirus AP-(Herpesviridae), Carajas virus (Rhabdoviridae), Caraparu virus (Bunyaviridae), Carey Island virus (Flaviviridae), Casphalia extranea densovirus (Parvoviridae), Catu virus (Bunyaviridae), Caviid herpesvirus ((Herpesviridae)), CbaAr virus (Bunyaviridae), Cebine herpesvirus (Herpesviridae), Cercopithecine herpesvirus (Herpesviridae), Cervid herpesvirus (Herpesviridae), CG-virus (Bunyaviridae), Chaco virus (Rhabdoviridae), Chagres virus (Bunyaviridae), Chamois contagious ecthyma virus (Poxviridae), Chandipura virus (Rhabdoviridae), Changuinola virus (Reoviridae), Charleville virus (Rhabdoviridae), Chelonid herpesvirus (Herpesviridae), Chelonid herpesvirus (Herpesvirzdae), Chelonid herpesvirus (Herpesviridae), Chenuda virus (Reoviridae), Chick syncytial virus (Retroviridae), Chicken anemia virus (Circoviridae), Chicken parvovirus (Parvoviridae), Chikungunya virus (Togaviridae), Chilibre virus (Bunyaviridae), Chim virus (Bunyaviridae), Chimpanzee herpesvirus (Herpesviridae), Chironomus attenuatus entomopoxvirus (Poxviridae), Chironomus luridus entomopoxvirus (Poxviridae), Chironomus plumosus erltomopoxvirus (Poxviridae), Chobar Gorge virus (Reoviridae), Choristoneura biennis entomopoxvirus (Poxviridae), Choristoneura conflicta entomopoxvirus (Poxviridae), Choristoneura diversuma entomopoxvirus (Poxviridae), Chorizagrotis auxiliars entomopoxvirus (Poxviridae), Chub reovirus Germany (Reoviridae), Ciconiid herpesvirus (Herpesviridae), Clo Mor virus (Bunyaviridae), CoAr-virus (Bunyaviridae), Coastal Plains virus (Rhabdoviridae), Cocal virus (Rhabdoviridae), Coital exanthema virus (Herpesviridae), ColAn-virus (Bunyaviridae), Colocasia bobone disease virus, (Rhabdoviridae), Colorado tick fever virus, (Reoviridae), Columbia SK virus, (Picornaviridae), Columbid herpesvirus, (Herpesviridae), Connecticut virus, (Rhabdoviridae), Contagious ecthyma virus, (Poxviridae), Contagious pustular dermatitis virus, (Poxviridae), Corfu virus, (Bunyaviridae), Corriparta virus, (Reoviridae), Cotia virus, (Poxviridae), Cowpox virus, (Poxviridae), Crimean-Congo hemorrhagic fever virus, (Bunyaviridae), CSIRO village virus, (Reoviridae), Cynara virus, (Rhabdoviridae), Cyprinid herpesvirus, (Herpesviridae), Dabakala virus, (Bunyaviridae), D′Aguilar virus, (Reoviridae), Dakar bat virus, (Flaviviridae), DakArk virus, (Rhabdoviridae), Deer papillomavirus, (Papovaviridae), Demodema boranensis entomopoxvirus, (Poxviridae), Dengue virus, (Flaviviridae), Dengue virus group, (Flaviviridae), Dependovirus, (Parvoviridae), Dera Ghazi Khan virus, (Bunyaviridae), Dera Ghazi Khan virus Group, (Bunyaviridae), Dermolepida albohirtum entomopoxvirus, (Poxviridae), Dhori virus, (Orthomyxoviridae), Diatraea saccharalis densovirus, (Parvoviridae), Dobrava-Belgrade virus, (Bunyaviridae), Dolphin distemper virus, (Paramyxoviridae), Dolphinpox virus, (Poxviridae), Douglas virus, (Bunyaviridae), Drosophila C virus, (Picornaviridae), Dry Tortugas virus, (Bunyaviridae), duck adenovirus, (Adenoviridae), Duck adenovirus, (Adenoviridae), Duck astrovirus, (Astroviridae), Duck hepatitis B virus, (Hepadnaviridae), Duck plague herpesvirus syn. anatid herpesvirus, (Herpesviridae), Dugbe virus, (Bunyaviridae), Duvenhage virus, (Rhabdoviridae), Eastern equine encephalitis virus, (Togaviridae), Ebola virus Filoviridae, Echinochloa hoj a blanca virus; Genus Tenuivirus, Echinochloa ragged stunt virus, (Reoviridae), ectromelia virus, (Poxviridae), Edge Hill virus, (Flaviviridae), Egtved virus syn. viral hemorrhagic septicemia virus, (Rhabdoviridae), Elapid herpesvirus, (Herpesviridae), Elephant loxondontal herpesvirus, (Herpesviridae), Elephant papillomavirus, (Papovaviridae), Elephantid herpesvirus, (Herpesviridae), Ellidaey virus, (Reoviridae), Embu virus, (Poxviridae), Encephalomyocarditis virus, (Picornaviridae), Enseada virus, (Bunyaviridae), Entamoeba virus, (Rhabdoviridae), Entebbe bat virus, (Flaviviridae), Epizootic hemorrhagic disease viruses, (Reoviridae), Epstein-Barr virus, (Herpesviridae), Equid herpesvirus, (Herpesviridae), Equid herpesvirus, (Nerpesviridae), Equid herpesvirus, (Herpesviridae), Equine abortion herpesvirus, (Herpesviridae), Equine adeno-associated virus, (Parvoviridae), Equine adenovirus, (Adenoviridae), Equine arteritis virus, (Arterivirus), Equine cytomegalovirus, (Herpesviridae), Equine encephalosis viruses, (Reoviridae), Equine herpesvirus, (Herpesviridae), Equine infectious anemia virus, (Retroviridae), Equine papillomavirus, (Papovaviridae), Equine rhinopneumonitis virus, (Herpesviridae), Equine rhinovirus, (Picornaviridae), Eret-virus, (Bunyaviridae), Erinaceid herpesvirus, (Herpesviridae), Erve virus, (Bunyaviridae), Erysimum latent virus, Tymovirus, Esocid herpesvirus, (Herpesviridae), Essaouira virus, (Reoviridae), Estero Real virus, (Bunyaviridae), Eubenangee virus, (Reoviridae), Euonymus fasciation virus, (Rhabdoviridae), European bat virus, (Rhabdoviridae), European brown hare syndrome virus, (Caliciviridae), European elk papillomavirus, (Papovaviridae), European ground squirrel cytomegalovirus, (Herpesviridae), European hedgehog herpesvirus, (Herpesviridae), Everglades virus, (Togaviridae), Eyach virus, (Reoviridae), Facey's Paddock virus, (Bunyaviridae), Falcon inclusion body disease, (Herpesviridae), Falconid herpesvirus, (Herpesviridae), Farallon virus, (Bunyaviridae), Felid herpesvirus, (Herpesviridae), Feline calicivirus, (Caliciviridae), Feline herpesvirus, (Herpesviridae), Feline immunodeficiency virus, (Retroviridae), Feline infectious peritonitis virus, (Coronaviridae), Feline leukemia virus, (Retroviridae), Feline parlleukopenia virus, (Parvoviridae), Feline parvovirus, (Parvoviridae), Feline syncytial virus, (Retroviridae), Feline viral rhinotracheitis virus, (Herpesviridae), Fetal rhesus kidney virus, (Papovaviridae), Field mouse herpesvirus, (Herpesviridae), Figulus subleavis entomopoxvirus, (Poxviridae), Fiji disease virus, (Reoviridae), Fin V-virus, (Bunyaviridae), Finkel-Biskis-Jinkins murine sarcoma virus, (Retroviridae), Flanders virus, (Rhabdoviridae), Flexal virus, (Arenaviridae), Flock house virus, Nodaviridae, Foot-and-mouth disease virus A, (Picornaviridae), Foot-and-mouth disease virus ASIA, (Picornaviridae), Foot-and-mouth disease virus, (Picornaviridae), Forecariah virus, (Bunyaviridae), Fort Morgan virus, (Togaviridae), Fort Sherman virus, (Bunyaviridae), Foula virus, (Reoviridae), Fowl adenoviruses, (Adenoviridae), Fowl calicivirus, (Caliciviridae), Fowlpox virus, (Poxviridae), Fraser Point virus, (Bunyaviridae), Friend murine leukemia virus, (Retroviridae), Frijoles virus, (Bunyaviridae), Frog herpesvirus, (Herpesviridae), Fromede virus, (Reoviridae), Fujinami sarcoma virus, (Retroviridae), Fukuoka virus, (Rhabdoviridae), Gabek Forest virus, (Bunyaviridae), Gadget's Gully virus, (Flaviviridae), Galleria mellonella densovirus, (Parvoviridae), Gallid herpesvirus, (Herpesviridae), Gamboa virus, (Bunyaviridae), Gan Gan virus, (Bunyaviridae), Garba virus, (Rhabdoviridae), Gardner-Arnstein feline sarcoma virus, (Retroviridae), Geochelone carbonaria herpesvirus, (Herpesviridae), Geochelone chilensis herpesvirus, (Herpesviridae), Geotrupes sylvaticus entomopoxvirus, (Poxviridae), Gerbera symptomless virus, (Rhabdoviridae), Germiston virus, (Bunyaviridae), Getah virus, (Togaviridae), Gibbon ape leukemia virus, (Retroviridae), Ginger chlorotic fleckvirus, Sobemovirus, Glycine mottle virus, Tombusviridae, Goat herpesvirus, (Herpesviridae), Goatpox virus, (Poxviridae), Goeldichironomus holoprasimus entomopoxvirus, (Poxviridae), Golden shiner reovirus, (Reoviridae), Gomoka virus, (Reoviridae), Gomphrena virus, (Rhabdoviridae), Gonometa virus, (Picornaviridae), Goose adenoviruses, (Adenoviridae), Goose parvovirus, (Parvoviridae), Gordil virus, (Bunyaviridae), Gorilla herpesvirus, (Herpesviridae), Gossas virus, (Rhabdoviridae), Grand Arbaud virus, (Bunyaviridae), Gray Lodge virus, (Rhabdoviridae), Gray patch disease agent of green sea turtle, (Herpesviridae), Great Island virus, (Reoviridae), Great Saltee Island virus, (Reoviridae), Great Saltee virus, (Bunyaviridae), Green iguana herpesvirus, (Herpesviridae), Green lizard herpesvirus, (Herpesviridae), Grey kangaroopox virus, (Poxviridae), Grimsey virus, (Reoviridae), Ground squirrel hepatitis B virus, (Hepadnaviridae), GroupA-K rotaviruses, (Reoviridae), Gruid herpesvirus, (Herpesviridae), GUU-virus, (Bunyaviridae), Guajara virus, (Bunyaviridae), Guama virus, (Bunyaviridae), Guanarito virus, (Arenaviridae), Guaratuba virus, (Bunyaviridae), Guaroa virus, (Bunyaviridae), Guinea pig cytomegalovirus, (Herpesviridae), Guinea pig herpesvirus, (Herpesviridae), Guinea pig type C oncovirus, (Retroviridae), Gumbo Limbo virus, (Bunyaviridae), Gurupi virus, (Reoviridae), H-virus, (Parvoviridae), H virus, (Bunyaviridae), Hamster herpesvirus, (Herpesviridae), Hamster polyomavirus, (Papovaviridae), Hantaan virus, (Bunyaviridae), Hanzalova virus, (Flaviviridae), Hardy-Zuckerman feline sarcoma virus, (Retroviridae), Hare fibroma virus, (Poxviridae), Hart Park virus, (Rhabdoviridae), Hartebeest herpesvirus, (Herpesviridae), Harvey murine sarcoma virus, (Retroviridae), Hazara virus, (Bunyaviridae), FIB virus, (Parvoviridae), Hepatitis virus, (Picomaviridae), Hepatitis virus, (Hepadnaviridae), Hepatitis virus, (Flaviviridae), Herpesvirus M, (Herpesviridae), Herpesvirus papio, (Herpesviridae), Herpesvirus platyrrhinae type, (Herpesviridae), Herpesvirus pottos, (Herpesviridae), Herpesvirus saimiri, (Herpesviridae), Herpesvirus salmonis, (Herpesviridae), Herpesvirus sanguinus, (Herpesviridae), Herpesvirus scophthalmus, (Herpesviridae), Herpesvirus sylvilagus, (Herpesviridae), Herpesvirus T, (Herpesviridae), Herpesvirus tarnarinus, (Herpesviridae), Highlands J virus, (Togaviridae), Hirame rhabdovirus, (Rhabdoviridae), Hog cholera virus, (Flaviviridae), HoJo virus, (Bunyaviridae), Hepatitis delta virus, Satellites, Deltavirus, Hsiung Kaplow herpesvirus, (Herpesviridae), Hepatitis E virus, (Caliciviridae), Hepatopancreatic parvo-like virus of shrimps, (Parvoviridae), Heron hepatitis B virus, (Hepadnaviridae), Herpes ateles, (Herpesviridae), Herpes simiae virus, (Herpesviridae), Herpes simplex virus, (Herpesviridae), Herpes virus B, (Herpesviridae), Herpesvirus aotus, (Herpesviridae), Herpesvirus ateles strain, (Herpesviridae), Herpesvirus cuniculi, (Herpesviridae), Herpesvirus cyclopsis, (Herpesviridae), Huacho virus, (Reoviridae), Hughes virus, (Bunyaviridae), Human adenoviruses, (Adenoviridae), Human astrovirus, (Astroviridae), Human calicivirus, (Caliciviridae), Human caliciviruses, (Caliciviridae), Human coronavirus E, (Coronaviridae), Human coronavuus OC, (Coronaviridae), Human coxsackievirus, (Picornaviridae), Human cytomegalovirus, (Herpesviridae), Human echovirus, (Picomaviridae), Human enterovirus, (Picornaviridae), Human foamy virus, (Retroviridae), Human herpesvirus, (Herpesviridae), Human herpesvirus, Nerpesviridae, Human herpesvirus, (Herpesviridae), Human immunodeficiency virus, (Retroviridae), Human papillomavirus, (Papovaviridae), Human parainfluenza virus, (Paramyxoviridae), Human poliovirus, (Picornaviridae), Human Respiratory Syncytial Virus, (Paramyxoviridae), Human rhinovirus, (Picomaviridae), Human spumavirus, (Retroviridae), Human T-lymphotropic virus, (Retroviridae), Humpty Doo virus, (Rhabdoviridae), HY-virus, (Bunyaviridae), Hypr virus, (Flaviviridae), Laco virus, (Bunyaviridae), Ibaraki virus, (Reoviridae), Icoaraci virus, (Bunyaviridae), Ictalurid herpesvirus, (Herpesviridae), Len virus, (Reoviridae), Ife virus, (Reoviridae), Iguanid herpesvirus, (Herpesviridae), Ilesha virus, (Bunyaviridae), Ilheus virus, (Flaviviridae), Inclusion body rhinitis virus, (Herpesviridae), Infectious bovine rhinotracheitis virus, (Herpesviridae), Infectious bursal disease virus, Bimaviridae, Infectious hematopoietic necrosis vuus, (Rhabdoviridae), Infectious laryngotracheitis virus, (Herpesviridae), Infectious pancreatic necrosis virus, Birnavirzdae, InfluenzaA virus (A/PR//(HN), (Orthomyxoviridae), Influenza B virus (B/Lee/), (Orthomyxoviridae), Influenza C virus (C/California/), (Orthomyxoviridae), Ingwavuma virus, (Bunyaviridae), Inini virus, (Bunyaviridae), Inkoo virus, (Bunyaviridae), Inner Frame virus, (Reoviridae), Ippy virus, (Arenaviridae), Irituia virus, (Reoviridae), Isfahan virus, (Rhabdoviridae), Israel turkey meningoencephalitis virus, (Flaviviridae), Issyk-Kul virus, (Bunyaviridae), itaituba virus, (Bunyaviridae), Itaporanga virus, (Bunyaviridae), ltaqui virus, (Bunyaviridae), ltimirirn virus, (Bunyaviridae), ltupiranga virus, (Reoviridae), Jaagsiekte virus, (Retroviridae), Jacareacanga virus, (Reoviridae), Jamanxi virus, (Reoviridae), Jamestown Canyon virus, (Bunyaviridae), Japanaut virus, (Reoviridae), Japanese encephalitis virus, (Flaviviridae), Jan virus, (Reoviridae), JC virus, (Papovaviridae), Joa virus, (Bunyaviridae), Joinjakaka virus, (Rhabdoviridae), Juan Diaz virus, (Bunyaviridae), Jugra virus, (Flaviviridae), Juncopox virus, (Poxviridae), Junin virus, (Arenaviridae), Junonia coenia densovirus, (Parvoviridae), Jurona virus, (Rhabdoviridae), Jutiapa virus, (Flaviviridae), K virus, (Papovaviridae), K virus, (Bunyaviridae), Kachemak Bay virus, (Bunyaviridae), Kadarn virus, (Flaviviridae), Kaeng Khoi virus, (Bunyaviridae), Kaikalur virus, (Bunyaviridae), Kairi virus, (Bunyaviridae), Kaisodi virus, (Bunyaviridae), Kala Iris virus, (Reoviridae), Kamese virus, (Rhabdoviridae), Karnmavanpettai virus, (Reoviridae), Kannamangalam virus, (Rhabdoviridae), Kao Shuan virus, (Bunyaviridae), Karimabad virus, (Bunyaviridae), Karshi virus, (Flaviviridae), Kasba virus, (Reoviridae), Kasokero virus, (Bunyaviridae), Kedougou virus, (Flaviviridae), Kemerovo virus, (Reoviridae), Kenai virus, (Reoviridae), Kennedya virus Y, Potyviridae, Kern Canyon virus, (Rhabdoviridae), Ketapang virus, (Bunyaviridae), Keterah virus, (Bunyaviridae), Keuraliba virus, (Rhabdoviridae), Keystone virus, (Bunyaviridae), Kharagysh virus, (Reoviridae), Khasan virus, (Bunyaviridae), Kilham rat virus, (Parvoviridae), Kimberley virus, (Rhabdoviridae), Kindia virus, (Reoviridae), Kinkajou herpesvirus, (Herpesviridae), Kirsten murine sarcoma virus, (Retroviridae), Kismayo virus, (Bunyaviridae), Klamath virus, (Rhabdoviridae), Kokobera virus, (Flaviviridae), Kolongo virus, (Rhabdoviridae), Koolpinyah virus, (Rhabdoviridae), Koongol virus, (Bunyaviridae), Kotonkan virus, (Rhabdoviridae), Koutango virus, (Flaviviridae), Kowanyama virus, (Bunyaviridae), Kumlinge virus, (Flaviviridae), Kunjin virus, (Flaviviridae), Kwatta virus, (Rhabdoviridae), Kyzylagach virus, (Togaviridae), La Crosse virus, (Bunyaviridae), La Joya virus, (Rhabdoviridae), La-Piedad-Michoacan-Mexico virus, (Paramyxoviridae), Lacertid herpesvirus, (Herpesviridae), Lactate dehydrogenase-elevating virus, (Arterivirus), Lagos bat virus, (Rhabdoviridae), Lake Clarendon virus, (Reoviridae), Lake Victoria cormorant herpesvirus, (Herpesviridae), Langat virus, Flaviviridae, Langur virus, (Retroviridae), Lanjan virus, (Bunyaviridae), Lapine parvovirus, (Parvoviridae), Las Maloyas virus, (Bunyaviridae), Lassa virus, (Arenaviridae), Lato river virus, (Tombusviridae), Le Dantec virus, (Rhabdoviridae), Leanyer virus, (Bunyaviridae), Lebombo virus, (Reoviridae), Lednice virus, (Bunyaviridae), Lee virus, (Bunyaviridae), Leporid herpesvirus, (Herpesviridae), Leucorrhinia dubia densovirus, (Parvoviridae), Lipovnik virus, (Reoviridae), Liverpool vervet monkey virus, (Herpesviridae), Llano Seco virus, (Reoviridae), Locusta migratona entomopoxvirus, (Poxviridae), Lokem virus, (Bunyaviridae), Lone Star virus, (Bunyaviridae), Lorisine herpesvirus, (Herpesviridae), Louping ill virus, Flaviviridae, Lucke frog herpesvirus, (Herpesviridae), Lum virus, (Parvoviridae), Lukuni virus, (Bunyaviridae), Lumpy skin disease virus, (Poxviridae), Lundy virus, (Reoviridae), Lymantria dubia densovirus, (Parvoviridae), Lymphocytic choriomeningitis virus, (Arenaviridae), Machupo virus, (Arenaviridae), Macropodid herpesvirus (Herpesviridae), Madrid virus, (Bunyaviridae), Maguari virus, (Bunyaviridae), Main Drain virus, (Bunyaviridae), Malakai virus, (Rhabdoviridae), Malignant catarrhal fever virus of European cattle, (Herpesviridae), Malpais Spring virus, (Rhabdoviridae), Malva silvestris virus, (Rhabdoviridae), Manawa virus, (Bunyaviridae), Manawatu virus, (Nodaviridae), Manitoba virus, (Rhabdoviridae), Manzanilla virus, (Bunyaviridae), Map turtle herpesvirus, (Herpesviridae), Mapputta virus, (Bunyaviridae), Maprik virus, (Bunyaviridae), Maraba virus, (Rhabdoviridae), Marburg virus, (Filoviridae), Marco virus, (Rhabdoviridae), Marek's disease herpesvirus, (Herpesviridae), Marituba virus, (Bunyaviridae), Marmodid herpesvirus, (Herpesviridae), Marmoset cytomegalovirus, (Herpesviridae), Marmoset herpesvirus, (Herpesviridae), Marmosetpox virus, (Poxviridae), Marrakai virus, (Reoviridae), Mason-Pfizer monkey virus, (Retroviridae), Masou salmon reovirus, (Reoviridae), Matruh virus, (Bunyaviridae), Matucare virus, (Reoviridae), Mayaro virus, (Togaviridae), Mboke virus, (Bunyaviridae), Meaban virus, (Flaviviridae), Measles (Edmonston) virus, (Paramyxoviridae), Medical Lake macaque herpesvirus, (Herpesviridae), Melanoplus sanguinipes entomopoxvirus, (Poxviridae), Melao virus, (Bunyaviridae), Meleagrid herpesvirus, (Herpesviridae), Melilotus latent virus, (Rhabdoviridae), Melolontha melolontha entomopoxvirus, (Poxviridae), Mengovirus, (Picornaviridae), Mermet virus, (Bunyaviridae), Mice minute virus, (Parvoviridae), Mice pneumotropic virus, (Papovaviridae), Microtus pennsylvanicus herpesvirus, (Herpesviridae), Middelburg virus, (Togaviridae), Miller's nodule virus, (Poxviridae), Mill Door virus, (Reoviridae), Minatitlan virus, (Bunyaviridae), Mink calicivirus, (Caliciviridae), Mink enteritis virus, (Parvoviridae), Minnal virus, (Reoviridae), Mirabilis mosaic virus, Caulimovirus, Mirim virus, (Bunyaviridae), Mitchell river virus, (Reoviridae), Mobala virus, (Arenaviridae), Modoc virus, (Flaviviridae), Moju virus, (Bunyaviridae), Mojui dos Campos virus, (Bunyaviridae), Mokola virus, (Rhabdoviridae), Molluscurn contagiosurn virus, (Poxviridae), Molluscurn-likepox virus, (Poxviridae), Moloney murine sarcoma virus, (Retroviridae), Moloney virus, (Retroviridae), Monkey pox virus, (Poxviridae), Mono Lake virus, (Reoviridae), Montana myotis leukoencephalitis virus, (Flaviviridae), Monte Dourado virus, (Reoviridae), Mopeia virus, (Arenaviridae), Moriche virus, (Bunyaviridae), Mosqueiro virus, (Rhabdoviridae), Mossuril virus, (Rhabdoviridae), Mount Elgon bat virus, (Rhabdoviridae), Mouse cytomegalovirus, (Herpesviridae), Mouse Elberfield virus, (Picornaviridae), Mouse herpesvirus strain, (Herpesviridae), Mouse mammary tumor virus, (Retroviridae), Mouse thymic herpesvirus, (Herpesviridae), Movar herpesvirus, (Herpesviridae), Mucambo virus, (Togaviridae), Mudjinbarry virus, (Reoviridae), Muir Springs virus, (Rhabdoviridae), Mule deerpox virus, (Poxviridae), Multimammate mouse papillomavirus, (Papovaviridae), Mumps virus, (Pararnyxoviridae), Murid herpesvirus, (Herpesviridae), Murine adenovirus, (Adenoviridae), Z murine adenovirus, (Adenoviridae), Murine hepatitis virus, (Coronaviridae), Murine herpesvirus, (Herpesviridae), Murine leukemia virus, (Retroviridae), Murine parainfluenza virus, (Pararnyxoviridae), Murine poliovirus, (Picornaviridae), Murine polyomavirus, (Papovaviridae), Murray Valley encephalitis virus, (Flaviviridae), Murre virus, (Bunyaviridae), Murutucu virus, (Bunyaviridae), Mykines virus, (Reoviridae), Mynahpox virus, (Poxviridae), Myxoma virus, (Poxviridae), Nairobi sheep disease virus, (Bunyaviridae), Naranj al virus, (Flaviviridae), Nasoule virus, (Rhabdoviridae), Navarro virus, (Rhabdoviridae), Ndelle virus, (Reoviridae), Ndumu virus, (Togaviridae), Neckar river virus, (Tombusviridae), Negishi virus, (Flaviviridae), Nelson Bay virus, New Minto virus, (Rhabdoviridae), Newcastle disease virus, (Paramyxoviridae), Ngaingan virus, (Rhabdoviridae), Ngari virus, (Bunyaviridae), Ngoupe virus, (Reoviridae), Nile crocodilepox virus, (Poxviridae), Nique virus, (Bunyaviridae), Nkolbisson virus, (Rhabdoviridae), Nola virus, (Bunyaviridae), North Clett virus, (Reoviridae), North End virus, (Reoviridae), Northern cereal mosaic virus, (Rhabdoviridae), Northern pike herpesvirus, (Herpesviridae), Northway virus, (Bunyaviridae), NorwaLk virus, (Caliciviridae), Ntaya virus, (Flaviviridae), Nugget virus, (Reoviridae), Nyabira virus, (Reoviridae), Nyamanini virus, Unassigned, Nyando virus, (Bunyaviridae), Oak-Vale virus, (Rhabdoviridae), Obodhiang virus, (Rhabdoviridae), Oceanside virus, (Bunyaviridae), Ockelbo virus, (Togaviridae), Odrenisrou virus, (Bunyaviridae), Oedaleus senegalensis entomopoxvirus, (Poxviridae), Oita virus, (Rhabdoviridae), Okhotskiy virus, (Reoviridae), Okola virus, (Bunyaviridae), Olifantsvlei virus, (Bunyaviridae), Omo virus, (Bunyaviridae), Omsk hemorrhagic fever virus, (Flaviviridae), Onchorhynchus masou herpesvirus, (Herpesviridae), O'nyong-nyong virus, (Togaviridae), Operophtera brumata entomopoxvirus, (Poxviridae), Orangutan herpesvirus, (Herpesviridae), Orf virus, (Poxviridae), Oriboca virus, (Bunyaviridae), Oriximina virus, (Bunyaviridae), Oropouche virus, (Bunyaviridae), Orungo virus, (Reoviridae), Oryctes rhinoceros virus, Unassigned, Ossa virus, (Bunyaviridae), Quango virus, (Rhabdoviridae), Oubi virus, (Bunyaviridae), Ourem virus, (Reoviridae), Ovine adeno-associated virus, (Parvoviridae), Ovine adenoviruses, (Adenoviridae), (Astroviridae), Ovine herpesvirus, (Herpesviridae), Ovine pulmonary adenocarcinoma virus, (Retroviridae), Owl hepatosplenitis herpesvirus, (Herpesviridae), P virus, (Bunyaviridae), Pacheco's disease virus, (Herpesviridae), Pacora virus, (Bunyaviridae), Pacui virus, (Bunyaviridae), Pahayokee virus, (Bunyaviridae), Palestina virus, (Bunyaviridae), Palyam virus, (Reoviridae), Pan herpesvirus, (Herpesviridae), Papio Epstein-Barr herpesvirus, (Herpesviridae), Para virus, (Bunyaviridae), Pararnushir virus, (Bunyaviridae), Parana virus, (Arenaviridae), Parapoxvirus of red deer in New Zealand, (Poxviridae), Paravaccinia virus, (Poxviridae), Parma wallaby herpesvirus, (Herpesviridae), Paroo river virus, (Reoviridae), Parrot herpesvirus, (Herpesviridae), Parry Creek virus, (Rhabdoviridae), Pata virus, (Reoviridae), Pates monkey herpesvirus pH delta, (Herpesviridae), Pathurn Thani virus, (Bunyaviridae), Patois virus, (Bunyaviridae), Peaton virus, (Bunyaviridae), Percid herpesvirus, (Herpesviridae), Perdicid herpesvirus, (Herpesviridae), Perinet virus, (Rhabdoviridae), Peripianata fuliginosa densovirus, (Parvoviridae), Peste-des-petits-ruminants virus, (Paramyxoviridae), Petevo virus, (Reoviridae), Phalacrocoracid herpesvirus, (Herpesviridae), Pheasant adenovirus, (Adenoviridae), Phnom-Penh bat virus, (Flaviviridae), Phocid herpesvirus, (Herpesviridae), Phocine (seal) distemper virus, (Paramyxoviridae), Pichinde virus, (Arenaviridae), Picola virus, (Reoviridae), Pieris rapae densovirus, (Parvoviridae), Pigeon herpesvirus, (Herpesviridae), Pigeonpox virus, (Poxviridae), Badnavirus Piry virus, (Rhabdoviridae), Pisurn virus, (Rhabdoviridae), Pixuna virus, (Togaviridae), Playas virus, (Bunyaviridae), Pleuronectid herpesvirus, (Nerpesviridae), Pneumonia virus of mice, (Paramyxoviridae), Pongine herpesvirus, (Herpesviridae), Pongola virus, (Bunyaviridae), Ponteves virus, (Bunyaviridae), Poovoot virus, (Reoviridae), Porcine adenoviruses, (Adenoviridae), Porcine astrovirus, (Astroviridae), Porcine circovirus, Circoviridae, Porcine enteric calicivirus, (Caliciviridae), Porcine enterovirus, (Picornaviridae), Porcine epidemic diarrhea virus, (Coronaviridae), Porcine hemagglutinating encephalomyelitis virus, (Coronaviridae), Porcine parvovirus, (Parvoviridae), Porcine respiratory and reproductive syndrome, (Arterivirus), Porcine rubulavirus, (Paramyxoviridae), Porcine transmissible gastroenteritis virus, (Coronaviridae), Porcine type C oncovirus, (Retroviridae), Porton virus, (Rhabdoviridae), Potosi virus, (Bunyaviridae), Powassan virus, (Flaviviridae), Precarious Point virus, (Bunyaviridae), Pretoria virus, (Bunyaviridae), Primate calicivirus, (Caliciviridae), Prospect Hill virus, (Bunyaviridae), Pseudaletia includens densovirus, (Parvoviridae), Pseudocowpox virus, (Poxviridae), Pseudolumpy skin disease virus, (Herpesviridae), Pseudorabies virus, (Herpesviridae), Psittacid herpesvirus, (Herpesviridae), Psittacinepox virus, (Poxviridae), Puchong virus, (Rhabdoviridae), Pueblo Viejo virus, (Bunyaviridae), Puffin Island virus, (Bunyaviridae), Punta Salinas virus, (Bunyaviridae), Punta Toro virus, (Bunyaviridae), Purus virus, (Reoviridae), Puumala virus, (Bunyaviridae), Qalyub virus, (Bunyaviridae), Quailpox virus, (Poxviridae), Quokkapox virus, (Poxviridae), Rabbit coronavirus, (Coronaviridae), Rabbit fibroma virus, (Poxviridae), Rabbit hemorrhagic disease virus, (Caliciviridae), Rabbit kidney vacuolating virus, (Papovaviridae), Rabbit oral papillomavirus, (Papovaviridae), Rabbitpox virus, (Poxviridae), Rabies virus, (Rhabdoviridae), Raccoon parvovirus, (Parvoviridae), Raccoonpox virus, (Poxviridae), Radi virus, (Rhabdoviridae), Rangifer tarandus herpesvirus, (Herpesviridae), Ranid herpesvirus, (Herpesviridae), Raphanus virus, (Rhabdoviridae), Rat coronavirus, (Coronaviridae), Rat cytomegalovirus, (Herpesviridae), Rat virus, R, (Parvoviridae), Raza virus, (Bunyaviridae), Razdan virus, (Bunyaviridae), Red deer herpesvirus, (Herpesviridae), Red kangaroopox virus, (Poxviridae), Reed Ranch virus, (Rhabdoviridae), herpesvirus, (Herpesviridae), Reindeer papillomavirus, (Papovaviridae), Reptile calicivirus, (Caliciviridae), Resistencia virus, (Bunyaviridae), Restan virus, (Bunyaviridae), Reticuloendotheliosis virus, (Retroviridae), Rhesus HHV-like virus, (Herpesviridae), Rhesus leukocyte associated herpesvirus strain, (Herpesviridae), Rhesus monkey cytomegalovirus, (Herpesviridae), Rhesus monkey papillomavirus, (Papovaviridae), Rheumatoid arthritis virus, (Parvoviridae), Rift Valley fever virus, (Bunyaviridae), Rinderpest virus, (Paramyxoviridae), Rio Bravo virus, (Flaviviridae), Rio Grande virus, (Bunyaviridae), RML virus, (Bunyaviridae), Rochambeau virus, (Rhabdoviridae), Rocio virus, (Flaviviridae), Ross River virus, (Togaviridae), Rost Islands virus, (Reoviridae), Rous sarcoma virus, (Retroviridae), Royal farm virus, (Flaviuiridae), RT parvovirus, (Parvoviridae), Rubella virus, (Togaviridae), Russian spring summer encephalitis virus, (Flaviviridae), S-virus, (Reoviridae), SA virus, (Herpesviridae), Sabio virus, (Arenaviridae), Sabo virus, (Bunyaviridae), Saboya virus, (Flaviviridae), Sacbrood virus, (Picornaviridae), Sagiyama virus, (Togaviridae), Saimiriine herpesvirus, (Herpesviridae), SaintAbb's Head virus, (Reoviridae), Saint-Floris virus, (Bunyaviridae), Sakhalin virus, (Bunyaviridae), Sal Viej a virus, (Flaviviridae), Salanga virus, (Bunyaviridae), Salangapox virus, (Poxviridae), Salehabad virus, (Bunyaviridae), Salmonid herpesvirus, (Herpesviridae), Salmonis virus, (Rhabdoviridae), Sambucus vein clearing virus, (Rhabdoviridae), SanAngelo virus, (Bunyaviridae), San Juan virus, (Bunyaviridae), San Miguel sealion virus, (Caliciviridae), San Perlita virus, (Flaviviridae), Sand rat nuclear inclusion agents, (Herpesviridae), Sandfly fever Naples virus, (Bunyaviridae), Sandfly fever Sicilian virus, (Bunyaviridae), Sandjimba virus, (Rhabdoviridae), Sango virus, (Bunyaviridae), Santa Rosa virus, (Bunyaviridae), Santarem virus, (Bunyaviridae), Sapphire U virus, (Bunyaviridae), Saraca virus, (Reoviridae), Sarracenia purpurea virus, (Rhabdoviridae), Sathuperi virus, (Bunyaviridae), Saumarez Reef virus, (Flaviviridae), Sawgrass virus, (Rhabdoviridae), Schistocerca gregaria entomopoxvirus, (Poxviridae), Sciurid herpesvirus, (Herpesviridae), Sciurid herpesvirus, (Herpesviridae), Sealpox virus, (Poxviridae), Seletar virus, (Reoviridae) Semliki Forest virus, (Togaviridae), Sena Madureira virus, (Rhabdoviridae), Sendai virus, (Paramyxoviridae), Seoul Virus, (Bunyaviridae), Sepik virus, (Flaviviridae), Serra do Navio virus, (Bunyaviridae), Shamonda virus, (Bunyaviridae), Shark River virus, (Bunyaviridae), Sheep associated malignant catarrhal fever of, (Herpesviridae), Sheep papillomavirus, (Papovaviridae), Sheep pulmonary adenomatosis associated herpesvirus, (Herpesviridae), Sheeppox virus, (Poxviridae), Shiant Islands virus, (Reoviridae), Shokwe virus, (Bunyaviridae), Shope fibroma virus, (Poxviridae), Shuni virus, (Bunyaviridae), Sibine fusca densovirus, (Parvoviridae), Sigma virus, (Rhabdoviridae), Sikte water-borne virus, (Tombusviridae), Silverwater virus, (Bunyaviridae), virus, (Bunyaviridae), Simian adenoviruses, (Adenoviridae), Simian agent virus, (Papovaviridae), Simian enterovirus, (Picornaviridae), Simian foamy virus, (Retroviridae), Simian hemorrhagic fever virus, (Arterivirus), Simian hepatitis A virus, (Picomaviridae), Simian immunodeficiency virus, (Retroviridae), Simian parainfluenza virus, (Paramyxoviridae), Simian rotavirus SA, (Reoviridae), Simian sarcoma virus, (Retroviridae), Simian T-lymphotropic virus, (Retroviridae), Simian type D virus, (Retroviridae), Simian vancella herpesvirus, (Herpesviridae), Simian virus, (Papovaviridae), Simulium vittatum densovirus, (Parvoviridae), Sindbis virus, (Togaviridae), Sixgun city virus, (Reoviridae), Skunkpox virus, (Poxviridae), Smelt reovirus, (Reoviridae), Snakehead rhabdovirus, (Rhabdoviridae), Snowshoe hare virus, (Bunyaviridae), Snyder-Theilen feline sarcoma virus, (Retroviridae), Sofyn virus, (Flaviviridae), Sokoluk virus, (Flaviviridae), Soldado virus, (Bunyaviridae), Somerville virus, (Reoviridae), Sparrowpox virus, (Poxviridae), Spectacled caimanpox virus, (Poxviridae), SPH virus, (Arenaviridae), Sphenicid herpesvirus, (Herpesviridae), Spider monkey herpesvirus, (Herpesviridae), Spondweni virus, (Flaviviridae), Spring viremia of carp virus, (Rhabdoviridae), Squirrel fibroma virus, (Poxviridae), Squirrel monkey herpesvirus, (Herpesviridae), Squirrel monkey retrovirus, (Retroviridae), SR-virus, (Bunyaviridae), Sripur virus, (Rhabdoviridae), StAbbs Head virus, (Bunyaviridae), St. Louis encephalitis virus, (Flaviviridae), Starlingpox virus, (Poxviridae), Stratford virus, (Flaviviridae), Strigid herpesvirus, (Herpesviridae), Striped bass reovirus, (Reoviridae), Striped Jack nervous necrosis virus, (Nodaviridae), Stump-tailed macaque virus, (Papovaviridae), Suid herpesvirus, (Herpesviridae), Sunday Canyon virus, (Bunyaviridae), Sweetwater Branch virus, (Rhabdoviridae), Swine cytomegalovirus, (Herpesviridae), Swine infertility and respiratory syndrome virus, (Arterivirus), Swinepox virus, (Poxviridae), Tacaiuma virus, (Bunyaviridae), Tacaribe virus, (Arenaviridae), Taggart virus, (Bunyaviridae), Tahyna virus, (Bunyaviridae), Tai virus, (Bunyaviridae), Taiassui virus, (Bunyaviridae), Tamana bat virus, (Flaviviridae), Tamdy virus, (Bunyaviridae), Tamiami virus, (Arenaviridae), Tanapox virus, (Poxviridae), Tanga virus, (Bunyaviridae), Tanjong Rabok virus, (Bunyaviridae), Taro bacilliform virus, (Badnavirus), Tataguine virus, (Bunyaviridae), Taterapox virus, (Poxviridae), Tehran virus, (Bunyaviridae), Telok Forest virus, (Bunyaviridae), Tembe virus, (Reoviridae), Tembusu virus, (Flaviviridae), Tench reovirus, (Reoviridae), Tensaw virus, (Bunyaviridae), Tephrosia symptomless virus, (Tombusviridae), Termeil virus, (Bunyaviridae), Tete virus, (Bunyaviridae), Thailand virus, (Bunyaviridae), Theiler's murine encephalomyelitis virus, (Picornaviridae), Thermoproteus virus, Lipothrixviridae, Thiafora virus, (Bunyaviridae), Thimiri virus, (Bunyaviridae), Thogoto virus, (Orthomyxoviridae), Thormodseyjarklettur virus, (Reoviridae), Thottapalayam virus, (Bunyaviridae), Tibrogargan virus, (Rhabdoviridae), Tick-borne encephalitis virus, (Flaviviridae), Tillamook virus, (Bunyaviridae), Tilligerry virus, (Reoviridae), Timbo virus, (Rhabdoviridae), Tilmboteua virus, (Bunyaviridae), Tilmaroo virus, (Bunyaviridae), Tindholmur virus, (Reoviridae), Tlacotalpan virus, (Bunyaviridae), Toscana virus, (Bunyaviridae), Tradescantia/Zebrina virus, Potyviridae, Trager duck spleen necrosis virus, (Retroviridae), Tree shrew adenovirus, (Adenoviridae), Tree shrew herpesvims, (Herpesviridae), Triatoma virus, (Picornaviridae), Tribec virus, (Reoviridae), Trivittatus virus, (Bunyaviridae), Trombetas virus, (Bunyaviridae), Trubanarnan virus, (Bunyaviridae), Tsuruse virus, (Bunyaviridae), Tucunduba virus, (Bunyaviridae), Tumor virus X, (Parvoviridae), Tupaia virus, (Rhabdoviridae), Tupaiid herpesvirus, (Herpesviridae), Turbot herpesvirus, (Herpesviridae), Turbot reovirus, (Reoviridae), Turkey adenoviruses, (Adenoviridae), Turkey coronavirus, (Coronaviridae), Turkey herpesvirus, (Herpesviridae), Turkey rhinotracheitis virus, (Paramyxoviridae), Turkeypox virus, (Poxviridae), Turlock virus, (Bunyaviridae), Turuna virus, (Bunyaviridae), Tyuleniy virus, (Flaviviridae) Uasin Gishu disease virus, (Poxviridae), Uganda S virus, (Flaviviridae), Ulcerative disease rhabdovirus, (Rhabdoviridae), Umatilla virus, (Reoviridae), Umbre virus, (Bunyaviridae), Una virus, (Togaviridae), Upolu virus, (Bunyaviridae), UR sarcoma virus, (Retroviridae), Urucuri virus, (Bunyaviridae), Usutu virus, (Flaviviridae), Uting a virus, (Bunyaviridae), Utive virus, (Bunyaviridae), Uukuniemi virus, (Bunyaviridae) Vaccinia subspecies, (Poxviridae), Vaccinia virus, (Poxviridae), Vaeroy virus, (Reoviridae), Varicella-zoster virus, (Herpesviridae), Variola virus, (Poxviridae), Vellore virus, (Reoviridae), Venezuelan equine encephalitis virus, (Togaviridae), Vesicular exanthema of swine virus, (Caliciviridae), Vesicular stomatitis Alagoas virus, Rkabdoviridae, Vesicular stomatitis Indiana virus, (Rhabdoviridae), Vesicular stomatitis New Jersey virus, (Rhabdoviridae), Vilyuisk virus, (Picornaviridae), Vinces virus, (Bunyaviridae), Viper retrovirus, (Retroviridae), Viral hemorrhagic septicemia virus, (Rhabdoviridae), Virgin River virus, (Bunyaviridae), Virus III, (Herpesviridae), Visna/maedi virus, (Retroviridae), Volepoxvirus, (Poxviridae), Wad Medani virus, (Reoviridae), Wallal virus, (Reoviridae), Walleye epidermal hyperplasia, (Herpesviridae), Wanowrie virus, (Bunyaviridae), Warrego virus, (Reoviridae), Weddel water-borne virus, Tombusviridae, Weldona virus, (Bunyaviridae), Wesselsbron virus, (Flaviviridae), West Nile virus, (Flaviviridae), Western equine encephalitis virus, (Togaviridae), Wexford virus, (Reoviridae), Whataroa virus, (Togaviridae), Wildbeest herpesvirus, (Herpesviridae), Witwatersrand virus, (Bunyaviridae), Wongal virus, (Bunyaviridae), Wongorr virus, (Reoviridae), Woodchuck hepatitis B virus, (Hepadnaviridae), Woodchuck herpesvirus marmota, (Herpesviridae), Woolly monkey sarcoma virus, (Retroviridae), Wound tumor virus, (Reoviridae), WVU virus, (Reoviridae), WW virus, (Reoviridae), Wyeomyia virus, (Bunyaviridae), Xiburema virus, (Rhabdoviridae), Xingu virus, (Bunyaviridae), Y sarcoma virus, (Retroviridae), Yaba monkey tumor virus, (Poxviridae), Yaba-virus, (Bunyaviridae), Yaba-virus, (Bunyaviridae), Yacaaba virus, (Bunyaviridae), Yaounde virus, (Flaviviridae), Yaquina Head virus, (Reoviridae), Yata virus, (Rhabdoviridae), Yellow fever virus, (Flaviviridae), Yogue virus, (Bunyaviridae), Yokapox virus, (Poxviridae), Yokase virus, (Flaviviridae), Yucca baciliform virus, Badnavirus, Yug Bogdanovac virus, (Rhabdoviridae), Zaliv Terpeniya virus, (Bunyaviridae), Zea mays virus, (Rhabdoviridae), Zegla virus, (Bunyaviridae), Zika virus, (Flaviviridae), Zirqa virus, (Bunyaviridae).

### Dosage Forms

The compositions as disclosed herein can provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a granule, a film, a sprinkle, a pellet, a bead, a pill, a powder, a triturate, a platelet, a strip or a sachet. Compositions can also be administered after being mixed with, for example yoghurt or fruit juice and swallowed or followed with a drink or beverage. These forms are well known in the art and are packaged appropriately. The compositions can be formulated for oral or rectal delivery.

Tablets prepared for oral administration according to the invention, and manufactured usmg direct compression, will generally contain other inactive additives such as binders, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate ( ), calcium stearate, stearic acid, and hydrogenated vegetable oil (preferably comprised of hydrogenated and refined triglycerides of stearic and palmitic acids at about 1 wt. % to 5 wt. %, most preferably less than about 2 wt. %). Lubricants may be present in a concentration of, for example, from about 0.25 wt. % to about 3 wt. %, 0.5 wt. % to about 2.0 wt. %, from about 0.75% to about 1.5%.

Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions. Disintegrants may be present in a concentration of, for example, from about 0.25 wt. % to about 3 wt. %, 0.5 wt. % to about 2.0 wt. %, from about 0.75% to about 1.5%.

Shellac, also called purified lac, a refined product obtained from the, resinous secretion of an insect. This coating dissolves in media of pH>7.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method as disclosed herein, the combination of the invention may be administered to mammalian species, such as dogs, cats, humans, etc. and as such may be incorporated in a conventional systemic dosage form, such as a tablet, capsule, or elixir. The above dosage forms will also include the necessary carrier material, excipient, viscosity modifier, lubricant, buffer, antibacterial, bulking agent (such as mannitol), anti-oxidants (ascorbic acid of sodium bisulfate) or the like.

The dose administered may be carefully adjusted according to age, weight and condition of the patient, as well as the route of administration, dosage form and regimen and the desired result.

The compositions of the invention may be administered in the dosage forms in single or divided doses of one to four times daily, or may be administered multiple times per day. It may be advisable to start a patient on a low dose combination and work up gradually to a high dose combination.

Tablets of various sizes can be prepared, e.g., of about 2 to 2000 mg in total weight, containing one or both of the active ingredients, with the remainder being a physiologically acceptable carrier of other materials according to accepted practice. Gelatin capsules can be similarly formulated.

Liquid formulations can also be prepared by dissolving or suspending one or the combination of active substances in a conventional liquid vehicle acceptable for administration so as to provide the desired dosage in, for example, one to four teaspoonfuls.

Dosage forms can be administered to the patient on a regimen of, for example, one, two, three, four, five, six, or other multiple doses per day.

In order to more finely regulate the dosage schedule, the active substances may be administered separately in individual dosage units at the same time or carefully coordinated times. The respective substances can be individually formulated in separate unit dosage forms in a manner similar to that described above.

In formulating the compositions, the active substances, in the amounts described above, may be compounded according to accepted practice with a physiologically acceptable vehicle, carrier, excipient, binder, viscosity modifier, preservative, stabilizer, flavor, etc., in the particular type of unit dosage form.

### Packaging/Freatment Kits

The disclosure provides a kit for conveniently and effectively carrying out the methods in accordance with the present disclosure. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging unit dosage forms. If desired, the blister can be in the form of a childproof blister, i.e. a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as, for example, an AM dose is packaged with a “midday” and a PM dose; or an AM dose is packaged with a PM dose. Alternatively, placebo dosages, or vitamin or dietary supplements, either in a form similar to or distinct from the active dosages, can be included.

The disclosure provides compositions, including preparations, formulations and/or kits, comprising combinations of ingredients, as described above (including the multi-ingredient combinations of drugs of the invention), that are serviceable as therapies for treating, preventing or improving conditions, states and disease as provided in the invention. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists two or more separate compartments. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the agents of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a combination of the invention), and then a “blister card” that is a printed card with an adhesive coating on the front surface. During the assembly process, the blister component, which is most commonly made out of PVC, is attached to the blister card using a blister machine. Conventional blister packs can also be sealed.

As discussed herein, the products of manufacture of the invention can comprise the packaging of the therapeutic drug combinations of the invention, alone or in combination, as “blister packages” or as a plurality of packets, including as lidded blister packages, lidded blister or blister card or packets, or a shrink wrap.

In one aspect, any of the invention's products of manufacture, including kits or blister packs, include memory aids to help remind patients when and how to take the agents of the invention.

The treatment kits can be constructed in a variety of forms familiar to one of ordinary skill in the art. The kits comprise at least one unit dosage of an active for administration according to a daily regimen and a means for containing the unit dosages. The treatment kits can, for example, be constructed for administration once daily, twice daily, thrice daily, four times daily, multiple administrations daily, or other dosage regimens. The kits comprise a means for the daily administration of an agent of the invention. In one embodiment the kits include from about one to about four unit dosages.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e., a second card or insert card, which is not permanently attached or affixed to the main card.

The memory aid can include a listing of the days of the week, i.e. Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (e.g.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

### Therapy

In some embodiments, treatment of subjects with a viral infection, such as COVID-19 or with a likelihood of developing COVID-19 using an effective amount of a diet and/or agents as disclosed herein increases the survival of the subjects. In further embodiments, the administration of an effective amount of a diet and/or agents as disclosed herein increases the survival of subjects with a viral infection, such as COVID-19 by at least 10%, 20%, 30%, 40%, 50%, 60%, 70%, 80%, 90% or 100% compared to a control group or historical controls. In other embodiments, treatment with an effective amount of an diet and/or agents as disclosed herein increases survival by at least 7 days, 14 days, 21 days, 28 days, 1 month, 2 months, 4 months, 6 months, or 12 months compared to a control group or historical controls. In additional embodiments, survival is measured after 1 week, 2 weeks, 3 weeks, 4 weeks, 30 days, 60 days, or 1 year post-hospitalization or post-intubation.

In other embodiments, the administration of a diet and/or agents as disclosed herein reduces the time needed on a ventilator by a patient, measured from time of intubation. In particular embodiments, administration of an diet and/or agents as disclosed herein reduces the time needed on a ventilator by a patient with a viral infection, such as COVID-19, by at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, or at least 4 weeks compared to a control group or historical controls. In further embodiments, treatment with a diet and/or agents as disclosed herein reduces the need for mechanical ventilation at least 5%, 10%, 15%, 20%, 25%, 30%, 35%, 40%, 45%, 50%, 55%, 60%, 65%, 70%, 75%, 80%, 90%, 95%, or 100% compared to a matched control group or historical controls. In other embodiments, treatment with a diet and/or agents as disclosed herein increased the proportion of subjects that never require mechanical ventilation by at least 5%, 10%, 15%, 20%, 25%, 30%, 35%, 40%, 45%, 50%, 55%, 60%, 65%, 70%, 75%, 80%, 90%, 95%, or 100% compared to a matched control group or historical controls.

In some embodiments, administration of diet and/or agents as disclosed herein, reduces the need for ECMO, compared to a matched control group or historical controls. In particular embodiments, treatment with a diet and/or agents as disclosed herein reduces the need for ECMO by at least 5%, 10%, 15%, 20%, 25%, 30%, 35%, 40%, 45%, 50%, 55%, 60%, 65%, 70%, 75%, 80%, 90%, 95%, or 100% compared to a matched control group or historical controls. In other embodiments, treatment with a diet and/or agents as disclosed herein reduces the days on mechanical ventilation or ECMO by at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days compared to a matched control group or historical controls.

As used herein, the terms “reduce,” “reducing,” or “reduction” in the context of treating a subject with a viral infection, such as COVID-19 refers to treatment that eases, mitigates, alleviates, ameliorate or decreases the effect or severity of a symptom of COVID-19, without curing the underlying disease, e.g., SARS-CoV-2 infection. Any indicia of success in reducing one or more symptoms of a viral infection, such as COVID-19 is recognized as reducing the symptoms. The reduction of a viral infection, such as COVID-19 symptom can be determined using standard routine clinical tests, radiologic or other imaging modalities and observations including ventilator settings, blood oxygenation levels, supplemental oxygen consumption that are well within the skill and knowledge of a medical professional. Other exemplary measurements or tests that can be used to monitor response to treatment include a reduced need for medications such as vasopressors, or the resolution of leukopenia.

In some embodiments, the methods of the invention reduce the occurrence or severity of symptoms of a viral infection, such as COVID-19, by at least 10%, at least 15%, at least 20%, at least 25%, at least 30%, at least 35%, at least 40%, at least 50%, at least 60%, at least 70%, at least 80%, or at least 90% compared to a baseline measurement or to a control group or historical controls.

In some embodiments, the administration of an effective amount of diet and/or agents as disclosed herein to a subject with a viral infection, such as COVID-19 reduces the extent of pulmonary exudate compared to a baseline measurement. Typically, the occurrence and extent of pulmonary exudate is determined using radiographic imaging techniques, such as chest x-rays or CT scans, but any suitable modality can be used. In some embodiments, the administration of a diet and/or agents as disclosed herein reduces the extent of pulmonary exudate by at 10%, at least 15%, at least 20%, at least 25%, at least 30%, at least 35%, at least 40%, at least 50%, at least 60%, at least 70%, at least 80%, or at least 90% compared to a baseline measurement.

In some embodiments, a method is provided for increasing, compared to a baseline measurement, the blood oxygenation level in a subject with a viral infection, such as COVID-19. The method comprises administering to the subject an diet and/or agents as disclosed herein, thereby increasing the subject's blood oxygenation level. In particular embodiments, blood oxygenation status is expressed as the ration PaO2/FiO2, where:
PaO2=Partial pressure of oxygen in arterial blood, in mmHg; and
FiO2=Fraction of inspired oxygen, in percent.

In further embodiments, the blood oxygenation level is measured using the oxygenation index (01), calculated as follows:

OI=[FiO2×mean airway pressure×IO0)/PaO2], and mean airway pressure is measured in mmHg.

The 01 measures the fraction of inspired oxygen (FiO2) and its usage within the body. A lower oxygenation index is better. As the oxygenation of a person improves, a higher PaO2 will be achieved at a lower FiO2, thus lowering the measured 01.

In other embodiments, the blood oxygenation level is measured using the oxygenation saturation index (OSI), calculated as follows:

OSI=[FiO2×mean airway pressure×I00)/oxygen saturation by pulse oximetry (SpO2)]

The OSI is a reliable noninvasive surrogate for the 01 that is associated with hospital mortality and ventilator-free days (VFDs) in patients with ARDS (DesPrez K, et al. Oxygenation Saturation Index Predicts Clinical Outcomes in ARDS. Chest. 2017 December; 152(6): 1151-1158).

In further embodiments, the blood oxygenation level is determined using pulse oximetry. This method measures peripheral oxygen saturation (SpO2), typically measured using a subject's finger, ear, or toe.

In some embodiments, administration of diet and/or agents as disclosed herein according to the methods of the invention, increases a subject's blood oxygenation level, as measured by PaO2/FiO2, 01, OSI, SpO2 or other suitable means, at least 5%, 10%, 15%, 20%, 25%, 30%, 35%, 40%, 45%, 50%, 55%, 60%, 65%, 70%, 75%, 80%, 90%, 95%, or 100% compared to a baseline measurement taken before the administration of the diet and/or agents as disclosed herein. In further embodiments, the comparison measurement is made about 24 h, 36 h, 48 h, 72 h, 96 h, 108 h, 120 h, 132 h, 148 h, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, or 24 days following initiation of diet and/or agents as disclosed herein therapy.

In other embodiments, administration of diet and/or agents as disclosed herein according to the methods of the invention, increases a subject's blood oxygenation level, as measured by PaO2/FiO2, 01, OSI, SpO2 or other suitable means, at least 5%, 10%, 15%, 20%, 25%, 30%, 35%, 40%, 45%, 50%, 55%, 60%, 65%, 70%, 75%, 80%, 90%, 95% or 100% compared to a match control group or historical controls. The blood oxygenation level measurement can be taken approximately 24 h, 36 h, 48 h, 72 h, 96 h, 108 h, 120 h, 132 h, 148 h, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days or 28 days following initial diagnosis of ARDS, initiation of treatment, or intubation for mechanical ventilation.

As used herein, the term “effective amount” in the context of administering an diet and/or agents as disclosed herein to a subject with a viral infection, such as COVID-19 or with the likelihood of developing COVID-19, refers to the amount of an diet and/or agents as disclosed herein that is sufficient to produce a beneficial or therapeutic effect including: the prevention of ARDS; the stabilization of one or more symptoms of the ARDS; the amelioration or reduction in the severity of one or more symptoms of COVID-19; an improvement in clinical status; or an increase in survival. In some embodiments, an “effective amount” of a diet and/or agents as disclosed herein refers to an amount of the diet and/or agents as disclosed herein that is sufficient to prevent the development of hypoxia, dyspenia, or other clinical symptoms of COVID-19. In further embodiments, the administration of an diet and/or agents as disclosed herein according to the methods of the invention prevent the further decline, slow the decline, or reverse the decline in blood oxygenation levels in a subject at risk for developing COVID-19. In additional embodiments, the diet and/or agents as disclosed herein is administered to a subject at risk for developing COVID-19 when the subject's blood oxygenation level, typically measured by pulse oximetry although any suitable means of measurement may be used, falls to below about 99%, 98%, 97%, 96%, 95%, 94%, 93%, 92%, 91%, 90%, 89%, 88%, 87%, 86%, or 85%.

Other clinical parameters that can be used to measure the health status of a subject with COVID-19 include serum creatinine (kidney function), serum bilirubin (liver function), platelet count (hematologic system function), Glasgow Coma Scale score (neurologic function), or the use of vasopressors (cardiovascular function).

In some embodiments, the clinical status of patients is assessed using an 8-point Modified WHO Ordinal Scale as specified below:
1. Not hospitalized, no limitations on activities
2. Not hospitalized, limitations on activities and/or requiring home oxygen
3. Hospitalized, not requiring supplemental oxygen, no longer requiring medical care
4. Hospitalized, not requiring supplemental oxygen, requiring ongoing medical care (COVID-19-related or otherwise)
5. Hospitalized, requiring supplemental oxygen
6. Hospitalized, requiring nasal high-flow oxygen, non-invasive mechanical ventilation, or both
7. Hospitalized, requiring invasive mechanical ventilation, extra-corporeal membrane oxygenation (ECMO), or both
8. Death

In some embodiments, treatment of subjects with ARDS with an diet and/or agents as disclosed herein improves their clinical status by at least 1 point, using the Modified WHO Ordinal Scale, over a matched control group or historical controls.

In other embodiments, treatment of subjects with a viral infection, such as COVID-19, with a diet and/or agents as disclosed herein hastens their recovery time compared to a matched control group or historical controls. Recovery time is defined as the first day on which a patient satisfies one of the following three categories from the ordinal scale: hospitalized, not requiring supplemental oxygen; not hospitalized (discharged), but with limitation on activities and/or requiring home oxygen; or not hospitalized (discharged), with no limitations on activities and not requiring supplemental oxygen. In additional embodiments, treatment of a subject with a viral infection, such as COVID-19, with a diet and/or agents as disclosed herein hastens the subject's recovery time, compared to a matched control group or historical controls, by at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days.

In other embodiments, treatment of subjects with a viral infection, such as COVID-19, with a diet and/or agents as disclosed herein reduces all-cause mortality by at least 5%, 10%, 15%, 20%, 25%, 30%, 35%, 40%, 45%, 50%, 55%, 60%, 65%, 70%, 75%, 80%, 90%, 95%, or 100% compared to a matched control group or historical controls. In further embodiments, treatment of subjects with a viral infection, such as COVID-19, with an diet and/or agents as disclosed herein reduces time in the ICU or CCU by at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or 28 days compared to a matched control group or historical controls. In additional embodiments, treatment of subjects with a viral infection, such as COVID-19 with a diet and/or agents as disclosed herein reduces hospitalization time by at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or 28 days compared to a matched control group or historical controls. In other embodiments, treatment of subjects with a viral infection, such as COVID-19 with a diet and/or agents as disclosed herein reduces supplemental oxygen consumption at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or 28 days compared to a matched control group or historical controls.

### Subjects

In some embodiments, subjects suitable for or in need of treatment with diet and/or agents as disclosed herein according to the methods of the present invention are mammals, more preferably humans, who are at risk of developing COVID-19, are experiencing hypoxia or dyspenia, or have already displayed at least one symptom of COVID-19. Subjects at risk for developing COVID-19 include subjects with a diagnosis of a bacterial or virological infection, including pulmonary infection. Typically, diagnosis is made by polymerase chain reaction (PCR) or culturing of a sample obtained, for example, from the subject's nasal passages, throat, mouth, sinuses, lungs, sputum, saliva or blood. Infectious agents associated with the development of ARDS include bacteria, particularly pneumococcia, mycoplasmas, and protozoans, and viruses, particularly, respiratory viruses that cause nosocomial or community-acquired viral pneumonia, including the Herpesviridae members herpes simplex virus (HSV) and cytomegalovirus (CMV). Other viruses associated with the development of ARDS include members of Coronaviridae, and, more specifically, members of the sub-family Orthocoronavirinae, also known as coronaviruses. Particular coronaviruses associated with the development of ARDS include SARS-CoV, HCoV NL63, HKUl, MERS-CoV, and SARS-CoV-2.

In some embodiments, a method of preventing ARDS is provided, the method comprising administering to a subject at risk of developing ARDS an effective amount of a diet and/or agents as disclosed herein, thereby preventing the development of ARDS. In particular embodiments, a method of preventing the development of ARDS in a subject with a SARS-CoV-2 infection is provided, the method comprises administering to the infected subject an effective amount of an diet and/or agents as disclosed herein, thereby preventing the development of ARDS in the subject. In further embodiments, a method of treating ARDS associated with an infection with SARS-CoV-2 is provided, the method comprises administering to a subject with ARDS, an effective amount of a diet and/or agents as disclosed herein, thereby treating the subject's ARDS.

COVID-19 may also develop as a consequence of sepsis, septic shock, or toxic shock following a non-pulmonary infection with a bacterium, fungus, protozoan or virus. Common non-pulmonary organ locations of the primary infection include brain, skin, urinary track and abdominal organs. More than 50% of cases of sepsis are the result of an infection with a gram-positive bacteria, most commonly staphylococci. Other commonly implicated bacteria include Streptococcus pyogenes, Escherichia coli, Pseudomonas aeruginosa, and Klebsiella species. Fungal sepsis accounts for approximately 5% of severe sepsis and septic shock cases; the most common cause of fungal sepsis is an infection by Candida species of yeast. In some embodiments, a method of preventing the development of ARDS in a subject with sepsis, septic shock or toxic shock is provided. The method comprises administering to the subject with sepsis, septic shock, or toxic shock an effective amount of an diet and/or agents as disclosed herein, thereby preventing the development of COVID-19. In further embodiments, a method of treating a subject with ARDS associated with sepsis, septic shock, or toxic shock is provided. The method comprises administering to the subject with COVID-19 associated with sepsis, septic shock, or toxic shock an effective amount of an diet and/or agents as disclosed herein, thereby treating the subject's ARDS.

Additional subjects at risk for developing COVID-19 include patients that experience various pulmonary injuries caused by non-infectious agents or factors, such as aspiration of gastric contents, near-drowning, blunt chest contusion, multiple injuries, inhalation burns, pancreatitis, and multiple blood transfusions. The risk of developing COVID-19 further increases when, in addition to any of the above listed causes, the subject also has advanced age (60 years), the presence of hypertension, diabetes, or other comorbidities. Subjects suspected of having ARDS can be readily identified by any competent medical practitioner using standard diagnostic tests and criteria including blood tests or radiographic imaging.

Patient characteristics associated with the likelihood of requiring ventilation include elevated lactate dehydrogenase, elevated high-sensitivity C-reactive protein, elevated interleukin-6, elevated D-dimer, and chest radiographic abnormalities (Wu C et al. JAMA Intern Med 2020 Mar. 13).

### Administration

The pharmaceutical compositions may be optimized for particular types of delivery. For example, pharmaceutical compositions for oral delivery are formulated using pharmaceutically acceptable carriers that are well known in the art. The carriers enable the agents in the composition to be formulated, for example, as a tablet, pill, capsule, solution, suspension, sustained release formulation; powder, liquid or gel for oral ingestion by the subject.

The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

Typically, the composition may be applied repeatedly for a sustained period of time topically on the part of the body to be treated, for example, the eyelids, eyebrows, skin or scalp. The dosage regimen will generally involve regular, such as daily, administration for a period of treatment of at least one month, or at least three months, or at least six months.

Alternatively, the composition may be applied intermittently, or in a pulsed manner. Accordingly, an alternative embodiment of the disclosure is to apply the composition on an intermittent or pulsed dosage schedule. For example, the composition of the disclosure may be used for two or more days, stopped, then restarted again at a time from between 2 weeks to 3 months later, and at even more long-spaced intervals in the case of the scalp.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Alternatively, the amount specified may be the amount administered as the average daily, average weekly, or average monthly dose.

The invention will be illustrated in more detail with reference to the following Examples, but it should be understood that the present invention is not deemed to be limited thereto.

### EXAMPLES

### Example 1

### Protocol Title Multicenter, Randomized, Double-Blind, Controlled Phase 2 Trial of Effects and Interactions of Minocycline, Mycophenolate, and Low-Purine Diet on SpO2 in Mild and Moderate COVID-19 Patients

Phase: Phase 2
Indication: Mild and Moderate COVID-19
Study Drugs: Low-dose mycophenolate mofetil (50 mg BID) and Low-dose minocycline (50 mg BID).
Study Design: 2×2×2 factorial design. This study consists of a screening visit, then a 10-day treatment period, followed by an end of study visit approximately day 28.
Study Period: Planned duration of study enrollment: 6 months
Planned duration of treatment period per subject: 10 days
Planned follow-up: Day 28
Study Population: 600 subjects, male and female; 18-74 years of age, with PCR and clinically diagnosed COVID-19. Patients have mild or moderate COVID-19.2,3
Endpoints: Primary Endpoint
   Change in SpO² from Baseline to Treatment Day 10.
   Secondary endpoints:
1. Percentage of patients with 1 point worsening category of illness compared to baseline WHO CPS on Day 7, 14 and 28.4
2. Symptoms severity resolution (lasting for at least 48 hours) of Covid-19
   time to symptom resolution
   proportion of subjects demonstrating symptom resolution via the symptom questionnaire (FDA Covid-19 symptom guidance)5 on Day 3, 7, 14 and 28
3. Occurrence of Adverse Events, Severe Adverse Events and abnormal laboratory results
4. Proportion of subjects admitted to the ICU due to Covid-19 by Day 28
5. Proportion of subjects requiring mechanical ventilation due to Covid-19 by Day 28
6. Proportion of subjects with all-cause death and Covid-19 related deaths by Day 28
7. Change in SpO2 from baseline to Day 28.

### Key Inclusion Criteria

1. Male or female
2. Positive nasopharyngeal swab PCR test for COVD-19
3. Diagnosis of mild or moderate COVID-19 up to 4 days after symptom onset.6
4. Willing to take a low-guanosine diet provided by the sponsor during the study
5. Negative pregnancy test before study treatment
6. Ability to swallow oral medications
7. Willing and able to sign the written informed consent

### Key Exclusion Criteria

1. Patients who are pregnant or may be pregnant
2. History of any hypersensitivity to minocycline or mycophenolate or their components.
3. Use of a tetracycline derivative or mycophenolate derivative for concurrent systemic disorder.
4. History of sensitivity to the sun.
5. Use of any investigational or non-registered drug or vaccine within 30 days preceding the first dose of the study drugs, or planned use during the study period
6. Concurrent or ongoing treatment for any systemic infection within 14 days of Day 0.
7. History of myocardial infarction, cerebrovascular accident, transient ischemic attack within 90 days of Day 0.
8. The following co-morbidities (or any other disease that might interfere with the study in the opinion of the investigator): immunosuppression, chronic obstructive pulmonary disease, acute or chronic renal failure, or current neoplasm.

### Notes:

1. This study entails three treatments: minocycline, mycophenolate, and a low-guanosine diet. The two drug treatments, minocycline and mycophenolate, will be double-blinded. If feasible, the low-guanosine diet will also be double blinded.
2. Patients are to be classified as per FDA Guidance at www.fda.gov/regulatory-information/search-fda-guidance-documents/assessing-covid-19-related-symptoms-outpatient-adult-and-adolescent-subjects-clinical-trials-drugs.
3. Patients will be managed at home or in the hospital, at the discretion of the treating physician.
4. See WHO COVID-19 Therapeutic Trial Synopsis at www.who.int/publications/i/item/covid-19-therapeutic-trial-synopsis.
5. To clarify, if the patient has a specimen taken for PCR testing on day 4, and the results are positive on Day 5, the subject will be admitted into the study.

### Example 2

### The LOGU Diet and Portion Size Instructions

We often think of the idea of “portion size” when we think of controlling how much food we eat for weight loss purposes. However, there are other uses for learning about portion sizes. Knowing what a portion size is important to be able to quantify, or count, how much food you're eating. With the LOGU (low guanosine) diet, knowing portion size will be important to help you eat to the amount of nucleotides you have been prescribed and fill out food logs for the dietitian to review.

For the LOGU diet a portion size (also sometimes just called “a portion” or “one”) is 100 grams of the food.

100 grams is most accurately measured by using a small food scale. If you do not have access to a food scale, there are other ways to estimate 100 g, but it will not be as accurate as weighing your food. A portion of 100 g is also a little less than half-a-cup or also equal to about 3.5 ounces.

Below are some tips for measuring portions using a scale, measuring cups, or estimating ounces. It will not be necessary to measure your foods forever, but it is necessary in the beginning until you are able to recognize a portion size on the LOGU diet.

### Food Scale Method:

A digital scale is preferred.
Place a piece of wax paper or clear plastic wrap on scale, place desired food on scale until it reads 100 g.
Count 1 portion for every 100 g (for example—400 g equals four portions).

### Measuring Cup Method:

100 g is about less than a ½ cup, although there is some difference between foods, which is what makes it less reliable.
Count 1 portion for every ½ cup (for example—2 cups equals four portions).

### Estimating Portion Sizes Method:

This is least reliable method but is useful when eating away from home.
Using your hand as a reference:
Food the size of the palm of your hand is about 3 ounces
Food the size of the bottom half of a closed fist is about a ½ cup
Food the size of the entire closed fist is about 1 cup.

### Example 3

TABLE 1

| Dairy Products/ | Fruits/ | Vegetables/ | | | Beverages/ |
|---|---|---|---|---|---|
| Proteins | Nuts/Seeds | Legumes | Starches | Fats | Miscellaneous |
| Bacon | Apple | Asparagus | Bread Rolls | Butter | Baking Powder |
| Brie Cheese | Avocado | Baked Potato | White Bread | Olive Oil | Baking Soda |
| Butter | Blueberries | Bamboo Shoots | White Cane | Margarine | Champagne |
| Cheddar Cheese | Brazil Nuts | Beet Root | Sugar | Mayonnaise | Chewing Gum |
| Cottage Cheese | Cranberries | Carrot | White Rice | Vegetable Oil | Cinzano Bianco |
| Edam Cheese | Gooseberries | Celeriac | (plain, white) | | Coffee |
| Egg Whites | Grapes | Celery Root | White Pasta | | Cola Soda |
| Enmentaler | (green, red) | Chicory | | | Gelatins |
| Cheese | Green Olives | Chinese Leaves | | | Pilsner Beer |
| Gouda Cheese | (marinated) | (Bok Choy) | | | Red Wine |
| Ice Cream | Honeydewj | Cucumber | | | Sugar Substitutes |
| (plain vanilla) | Kiwi | Eggplant | | | (excluding Stevia) |
| Margarine | Morello Cherries | Endive | | | Tea |
| Quark | Orange | Fennel | | | Vinegar |
| Whole Milk | Peach | Iceberg Lettuce | | | Whey Protein |
| Yogurt | Pear | Kohlrabi | | | Isolate |
| | Pineapple | Lettuce | | | (vanilla) |
| | Plum | Onion | | | White Wine |
| | Quince | Radishes | | | |
| | Raspberries | Sauerkraut | | | |
| | Red Currants | (canned, drained) | | | |
| | Rhubarb | Summer Squash | | | |
| | Strawberries | Tomato | | | |
| | Sweet Cherries | Watercress | | | |
| | Walnuts | White Cabbage | | | |
| | | Zucchini | | | |

### Example 4

### Breakfast

Omelet of Egg Whites, Onion, Tomato, Cheddar Cheese, with Coffee, Milk 3 Egg Whites, 0 mg; Onion 7/16c, 13 mg; Tomato 7/16c, 11 mg; Cheddar Cheese 3.5 ounces, 7 mg.
Cottage Cheese with Peaches, Hot Tea
Cottage Cheese 7 ounces, 18 mg; Peaches 7/16 c 21 mg; Hot tea 7 ounces, 4 mg.
Fruited Yogurt with Brazil Nuts, with Hot Tea
Yogurt 7 ounces, 16 mg; Brazil Nuts 3.5 ounces, 23 mg.

### Lunch

Waldorf-style Salad with Iceberg Lettuce, Apples, Walnuts, Grapes, Mayonnaise, White Bread Roll
Iceberg Lettuce 14/16c 20 mg; Apple 7/16c 14 mg; Walnuts 3.5 ounces 26 mg; Grapes 7/16c 27 mg; Mayonnaise 3.5 ounces, 20 mg; White Bread Roll 1 small, 21 mg.
BLT Sandwich of White Bread, Bacon, Iceberg Lettuce, Tomato, Mayonnaise with Cucumber Slices
White Bread 2 slices, 28 mg; Bacon 3.5 ounces, 10; Lettuce 7/16c, 13 mg; Tomato Slices 7/16c, 11 mg; Cucumber Slices 14/16c, 14 mg.
Grilled Cheese Sandwich of White Bread, Edam Cheese, Margarine, with Cabbage Cole Slaw of White Cabbage, Mayonnaise, and Vinegar
White Bread 2 slices, 28 mg; Edam Cheese 7 ounces, 14 mg; Margarine 3/16c, 0 mg;
White Cabbage 14/16c, 44 mg; Vinegar 0 mg.

### Dinner

Pasta Primavera of White Pasta Noodles Eggplant, Tomato, Green Olives, Butter with Red Wine, Fresh Orange
White Pasta Noodles 14/16c, XXmg; Eggplant 7/16c, 21 mg; Tomato 7/16c, 11 mg; green Olives 7/16c, 29 mg; Butter 0 mg; Red Wine, 0 mg; Orange 7/16c, 19 mg.
Stir-fry of Rice, Carrots, Onion, Egg Whites, Bamboo Shoots with White Wine,
Vanilla Ice Cream
White Rice 14/16c XXmg; Carrots 7/16c, 17 mg; Onion 7/16c, 13 mg; 2 Egg Whites, 0 mg; Bamboo Shoots 7/16c, 29 mg; White Wine 0 mg; Ice Cream 7/16c, 40 mg.
Spanish Frittata of Egg Whites, Potato, Fennel Fronds with White Bread Toast, Red Wine, Raspberries with White Sugar
3 Egg Whites, 0 mg; Potato 7/16c 18 mg; Fennel Fronds 7/16c 14 mg; White Bread 1 slice, 14 mg; Red Wine 0 mg; Raspberries 7/16c, 18 mg.

### Snacks

Homemade White Bread Crostini with Brie Cheese
White Bread Roll 1 small, 21 mg; Brie Cheese 3.5 ounces, 7 mg.
Honeydew Melon Slices
Honeydew Melon 14/16c, 50 mg
Sweet Cherries
Sweet Cherries, 14/16c, 34 mg.

### Example 5

### Baseline Severity Categorization

SARS-CoV-2 infection without symptoms
Positive testing by virologic test (i.e., a nucleic acid amplification test of an antigen test)
No symptoms

### Mild COVID-19

Positive testing by virologic test (i.e., a nucleic acid amplification test of an antigen test)
Symptoms of mild illness with COVID-19 that could include fever, cough, sore throat, malaise, headache, muscle pain, nausea, vomiting, diarrhea, and loss of taste or smell, without shortness of breath or dyspnea
No clinical signs indicative of Moderate, Severe, or Critical Severity

### Moderate COVID-19

Positive testing by virologic test (i.e., a nucleic acid amplification test of an antigen test)
Symptoms of moderate illness with COVID-19, which could include any symptom of mild illness or shortness of breath with exertion
Clinical signs suggestive of moderate illness with COVID-19, such as respiratory rate 2: 20 breaths per minute, heart rate 2: 90 beats per minute; with saturation of oxygen (SpO2)>93% on room air at sea level
No clinical signs indicative of Severe or Critical Illness Severity

### Severe COVID-19

Positive testing by standard RT-PCR assay or an equivalent test
Symptoms suggestive of severe systemic illness with COVID-19, which could include any symptom of moderate illness or shortness of breath at rest, or respiratory distress
Clinical signs indicative of severe systemic illness with COVID-19, such as respiratory rate 2: 30 per minute, heart rate 2: 125 per minute, SpO²:′·S 93% on room air at sea level or PaO2/FiO2<300
No criteria for Critical Severity

### Critical COVID-19

Positive testing by virologic test (i.e., a nucleic acid amplification test of an antigen test)
Evidence of critical illness, defined by at least one of the following:
Respiratory failure defined based on resource utilization requiring at least one of the following:
Endotracheal intubation and mechanical ventilation, oxygen delivered by high-flow nasal cannula (heated, humidified, oxygen delivered via reinforced nasal cannula at flow rates >20 Umin with fraction of delivered oxygen 2: 0.5). noninvasive positive pressure ventilation, ECMO, or clinical diagnosis of respiratory failure (i.e., clinical need for one of the preceding therapies, but preceding therapies not able to be administered in setting of resource limitation)
Shock (defined by systolic blood pressure <90 mm Hg, or diastolic blood pressure <60 mm Hg or requiring vasopressors)
Multi-organ dysfunction/failure

NOTE: A clinical diagnosis of respiratory failure (in the setting of resource limitation) in which the management deviates from standard of care should be recorded as part of formal data collection.

### Example 5

Treatment of Covid-19 with Minocycline and a Guanosine-Restricted Diet.

A 21-year-old female Covid-19 patient was treated using the compositions and methods as disclosed herein, using a novel combination of minocycline and a guanosine-restricted diet. Minocycline is an antibiotic with broad spectrum anti-viral effects. Deprivation of guanosine is an effective anti-viral modality in vitro and in animal models. The patient's symptoms resolved rapidly.

A 21-year-old white female university student was told that her roommate tested positive for Covid, and two days later she developed fever and body aches. The patient took a PCR Covid test on that day and the positive results were reported to her four days later. Her medical history was unremarkable except for polycystic ovary disease for which she was had been prescribed a birth control pill. She was started on a 24 hour fast, in which no food was permitted but she was able to drink either water or milk (which is a protein; commercial milks are thought to contain no nucleic acids). She kept a food diary and a symptom diary. After 24 hours, she began a very low guanosine diet (called the Urgent diet). A few days later she was moved to a diet with somewhat more guanosine, and which is more palatable (called the Phase I diet). On day 4, she was started on minocycline 50 mg BID. About 36 hours after starting the minocycline she was asked how she felt, and she responded, “I feel great!” Upon further questioning, she indicated that her fever and body aches were gone and she had about 90% of her normal energy back. On day 5, she developed what she described as “swollen glands” on the neck and said that she always, her whole life, developed such signs when she had a cold. From her description it appears these were submandibular and superficial cervical lymph nodes. She said she typically takes acetaminophen and that the condition usually resolved in a few days. The “swollen glands” resolved by day 8. A summary of her history of symptoms is as follows: sore throat (days 1-3), fever (days 1-5), chills (days 1-2), body aches (days 1-5), headache (days 2-5), cough (days 2-4), fatigue (days 1-6), sinus congestion (days 1-8), “swollen glands” (days 5-8). The patient did not experience: loss of taste or smell, shortness of breath, nausea, or vomiting. She continued on the Phase 1 diet and minocycline to day 14. The patient took acetaminophen on days 1-5 (650 mg Q4H on days 1 and 2; 650 mg BID on days 3 and 4; 650 mg once on Day 5), and only disclosed this to the treating physician after two weeks. The patient's dietary intake of guanosine was about zero during the first day (the food fast), and about 15-35 mg/day for days 2-4 (the “Urgent” diet) and about 35-75 mg/day for days 5-14 (the “Phase 1” diet). For comparison, generally Americans take in about 2,000 mg/day of nucleotides, and we estimate therefore that they take in about 500 mg/day of guanosine. Thus, on any day of treatment, the patient took in 15% or less of the guanosine in a typical diet.

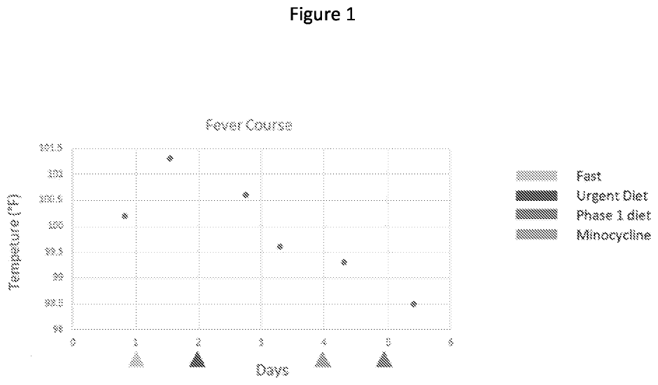

The patient had dramatic resolution of her fever and body aches, and the other symptoms quickly dissipated. (See FIG. 1). She had no further symptoms at all after day 8. It seems likely that the guanosine-restricted diet was responsible for this quick improvement. The acetaminophen she took may have reduced the height of her fever, but is unlikely to have caused the rapid decline and resolution of it and the other symptoms. The documented anti-viral, anti-inflammatory, and neuroprotective properties of minocycline likely contributed to her quick recovery.

This Covid-19 patient responded with rapid resolution of her fever and symptoms to a low-guanosine diet, which is believed to have inhibited replication of the virus, and she also appears to have benefited from the addition of treatment with minocycline.

Reduction of the pool of guanosine in cell and animal models of viral infection has been effective in mitigating signs and mortality from viral diseases. Evidence of reduction both by dietary means and by inhibiting enzymatic pathways that produce guanosine has been reported. Enzymatic pathway inhibition was specifically effective in treatment of SARS-CoV-2 in both in vitro and in animal models. The current Example indicates that dietary restriction of guanosine intake may also contribute to mitigation of Covid-19 replication and resolution of symptoms. As can be seen from FIG. 1, the rapid defervescence of the patient occurred when she was on low guanosine diet and before minocycline was started. Interestingly, a small group of MERS coronavirus patients treated with mycophenolate mofetil, an inhibitor of guanosine synthesis, did well, with no mortality.

### Example 6

A 55-year-old white female became PCR-positive for COVID in May 2022. She had received both doses of the Pfizer covid-19 vaccine and received the Pfizer booster in April 2022. Her initial symptoms included fever of 100.1° F., sore throat, chills, body aches, headache, cough, fatigue, and nasal congestion. At that time, she had no shortness of breath or loss of smell. She did say the taste of food was less than normal. There was no nausea or vomiting. She was started on minocycline 50 mg twice a day and mycophenolate mofetil 50 mg twice a day. She was instructed to be on a food fast (no food, only water) for 3 days, and then was started on a low-guanosine diet, which she adhered to for 12 days. She kept a symptom diary and food diary. The food diary confirmed that she was compliant with the diet instructions.

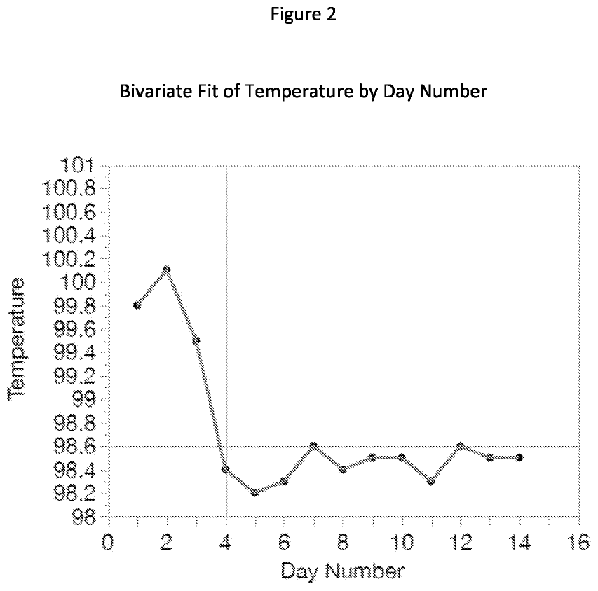

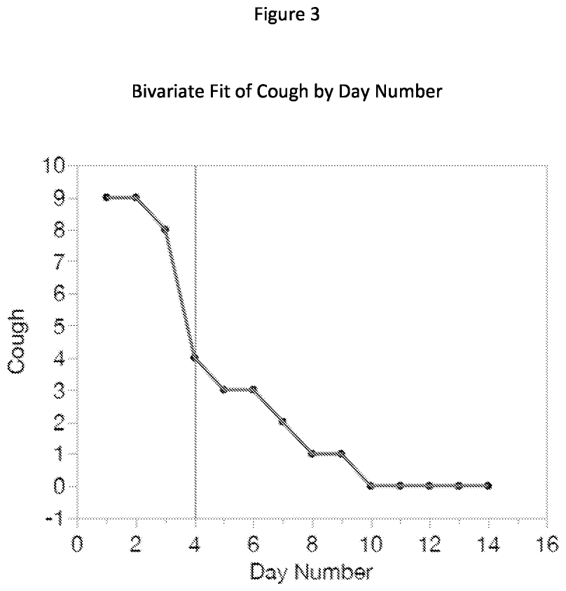

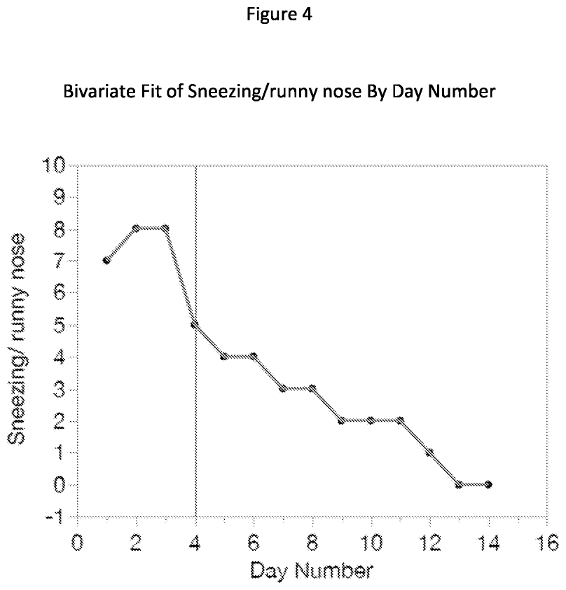

FIGS. 2-4 diagrams several of the patient's symptoms from her diary. After three days there was a dramatic improvement in her condition with respect to essentially all of the symptoms, so in each graph a reference line is placed at day 4, during which most symptoms had improved. (The temperature graph also has a horizontal reference line at normal, which is generally accepted to be at 98.6° F.) Table 2 summarizes some of the important data described below.

Her temperature was a maximum of 100.1° F. on day 2, first declined on day 3 to 99.5° F., and was first normal on day 4.

The statements below refer to all the symptoms, which were scored by the patient either 0 (symptom not present) or by severity (1 is least severe, 10 is most severe). All symptoms showed the first decline on days 3 or 4 (excluding loss of smell which was never present). The following symptoms were normal on day 4: temperature, sore throat, headache, loste of taste, nausea and vomiting. The following symptoms normalized on days 6-10: chills, swollen glands, body aches, and cough. The following symptoms normalized on days 12 or 13: fatigue, congestion, and sneezing/runny nose. Shortness of breath remained at a very low level (1) during that two week. The most severe symptoms (e.g. scores 8-9) all first declined on day 3, except for sneezing/runny nose which declined on day 4. Almost all the other symptoms first declined on day 4. In general, the most severe symptoms (score 9) took the longest to normalize (days 10-13).

TABLE 2

| | | on | Day of | to value | First |
|---|---|---|---|---|---|
| | Maximum | days | first decline | of | Normal Day |
| Temperature | 100.1 | 2 | 3 | 99.5 | 4 |
| Cough | 9 | 1-2 | 3 | 8 | 10 |
| Fatigue | 9 | 1-2 | 3 | 7 | 12 |
| Congestion | 9 | 2 | 3 | 8 | 13 |
| Sore Throat | 8 | 1-2 | 3 | 6 | 4 |
| Swollen Glands | 8 | 1-2 | 3 | 7 | 7 |
| Sneezing/ | 8 | 2-3 | 4 | 5 | 13 |
| RunnyNose | | | | | |
| Headache | 7 | 1-2 | 4 | 0 | 4 |
| Chills | 6 | 1-3 | 4 | 3 | 6 |
| Body Aches | 6 | 1-3 | 4 | 2 | 9 |
| Vomiting | 5 | 3 | 4 | 0 | 4 |
| Nausea | 2 | 3 | 4 | 0 | 4 |
| Shortness of | 1 | 1-2 | 3 | 0 | — |
| Breath | | | | | |
| Loss of Taste | 1 | 1-3 | 4 | 0 | 4 |
| Loss of Smell | 0 | all | | | |

In summary, the treatments described, which included mycophenolate, minocycline, and guanosine restriction diet, led to dramatic and rapid improvement in this Covid-19 patient's fever and symptoms.

About six months later, the patient, a singer, was reinfected when numerous members of her singing group tested positive for covid. The patient notified her physician the second week in November, 2022 that she had been exposed to people who became covid positive. She started on a food fast (only water permitted) that day (Day #1) and started the next day (Day #2) on the same regimen of minocycline and mycophenolate as previously. She tested positive for covid on Day #3. Her symptoms were much milder than the previous bout, and mainly included mild sore throat. fatigue, and headache. She felt much better on the fourth day after she started the medication (Day #6), and the symptoms were almost gone by the eighth day after starting the medication (Day #10). After 3 days on the food fast, she switched to the low-guanosine diet. She continued the medication and diet to a total of 14 days.

Summarizing this second infection episode, when this patient was exposed to covid-positive people, and then started the medications and low-guanosine diet a few days before she tested positive, she had milder symptoms which improved or resolved quickly.

While the invention has been described in detail and with reference to specific examples thereof, it will be apparent to one skilled in the art that various changes and modifications can be made therein without departing from the spirit and scope thereof.

1. A treatment method for improving, compared to a baseline measurement, the blood oxygenation level of a subject diagnosed with a viral infection, the treatment method comprising:
selecting a subject diagnosed with a viral infection and in need of improving, compared to a baseline measurement, blood oxygenation level;
administering to the patient at least one agent which depletes guanosine-containing nucleosides and nucleotides,
thereby improving the subject's blood oxygenation level.

2. The method of claim 1, wherein the at least one agent which depletes guanosine-containing nucleosides and nucleotides is selected from the group consisting of mycophenolate, IMPDH enzyme inhibitors, agents which bind to guanine, or guanosine-containing nucleosides and nucleotides, and combinations thereof.

3. The method of claim 1, wherein the baseline measurement is determined prior to infection, early in the subject's infection, from a standard, compared to a control group, or an historical control value.

4. The method of claim 1, wherein the subject is further diagnosed with acute respiratory distress syndrome (ARDS).

5. The method of claim 1, wherein the blood oxygenation level is determined using the oxygenation index, the oxygenation saturation index, pulse oximetry, and combinations thereof.

6. The method of claim 1, wherein the blood oxygenation level is determined at a time point selected from the group consisting of one day, two days, three days, four days, five days, six days, seven days, eight days, nine days, 10 days, 11 days, 12 days, 13 days, two weeks, three weeks, one month, two months, and three months.

7. The method of claim 1, wherein said treatment reduces ventilator use, reduces ECMO use, reduces time in the ICU or CCU, reduces hospitalization time, reduces all-cause mortality, or reduces supplemental oxygen consumption compared to a control group or historical controls.

8. The method of, wherein said treatment reduces pulmonary exudate levels or reduces supplemental oxygen consumption compared to a baseline measurement.

9. The method of claim 1, wherein said treatment increases subject's survival compared to a control group or historical controls.

10. The method of claim 1, wherein the viral infection is a coronavirus selected from the group consisting of SARS-CoV, HCoV NL63, HKU1, MERS-CoV, and SARS-CoV-2.

11. The method of claim 1, wherein the coronavirus is SARS-CoV-2.

12. A treatment method for improving, compared to a baseline measurement, the blood oxygenation level of a subject diagnosed with a viral infection, the treatment method comprising:
selecting a subject diagnosed with a viral infection and in need of improving, compared to a baseline measurement, blood oxygenation level;
administering to the patient a diet which depletes guanosine-containing nucleosides and nucleotides;
wherein the subject's blood oxygenation level is improved compared to a baseline measurement.

13. The method of claim 12, wherein the diet which depletes guanosine-containing nucleosides and nucleotides contains a nucleotide content which is selected from the group consisting of about 1000 mg/day, of about 750 mg/day, of about 500 mg/day, of about 250 mg/day, of about 100 mg/day, of about 75 mg/day, of about 50 mg/day, and of about 25 mg/day of nucleotide.

14. The method of claim 12, wherein the baseline measurement is determined prior to infection, early in the subject's infection, from a standard, compared to a control group, or an historical control value.

15. The method of claim 12, wherein the subject is further diagnosed with acute respiratory distress syndrome (ARDS).

16. The method of claim 12, wherein the blood oxygenation level is determined using the oxygenation index, the oxygenation saturation index, pulse oximetry, and combinations thereof.

17. The method of claim 12, wherein the blood oxygenation level is determined at a time point selected from the group consisting of one day, two days, three days, four days, five days, six days, seven days, eight days, nine days, 10 days, 11 days, 12 days, 13 days, two weeks, three weeks, one month, two months, and three months.

18. The method of claim 12, wherein said treatment reduces ventilator use, reduces ECMO use, reduces time in the ICU or CCU, reduces hospitalization time, reduces all-cause mortality, or reduces supplemental oxygen consumption compared to a control group or historical controls.

19. The method of claim 12, wherein said treatment reduces pulmonary exudate levels or reduces supplemental oxygen consumption compared to a baseline measurement.

20. The method of claim 12, wherein said treatment increases subject's survival compared to a control group or historical controls.

21. The method of claim 12, wherein the viral infection is a coronavirus selected from the group consisting of SARS-CoV, HCoV NL63, HKU1, MERS-CoV, and SARS-CoV-2.

22. The method of claim 12, wherein the coronavirus is SARS-CoV-2.

23-89. (canceled)
